SEQUENCE LISTING (3 sequences)

SEQ ID NO: 1 - protein, 103 aa, Sus scrofa
MESEEMLAGGLTEPRPATPEIQEIANKVKPQLEEKTNKTYEKFEAIIYRSQVVAGTNYYIKVHVGGNNYVHIRVFQSLPHQEDPLKLIGYQVDKTKDDELTGF

SEQ ID NO: 2 - DNA, 3768 bp, SARS coronavirus, Urbani strain
  misc_feature (1)..(3768): nucleotides 21492-25259 of GenBank Accession No. AY278741
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 3 - DNA, 2031 bp, Zaire ebolavirus
  misc_feature (1)..(2031): nucleotides 6041-8071 of Genbank Accession No. L11365
  sig_peptide (1)..(96)
atgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

(19) United States
(12) Patent Application Publication
(10) Pub. No.: US 2007/0203073 A1
(43) Pub. Date: Aug. 30, 2007

(54) SARS and Ebola inhibitors and use thereof, and methods for their discovery

(76) Inventors: Scott L. Diamond, Bala Cynwyd, PA (US); Dhaval Gosalia, Lansdale, PA (US); Graham Simmons, San Francisco, CA (US); Paul Bates, Swarthmore, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/472,167

(22) Filed: Jun. 21, 2006

Related U.S. Application Data
(60) Provisional application No. 60/693,028, filed on Jun. 22, 2005.

(51) Int. Cl. A61K 38/05; A61K 38/04; A61K 31/541; A61K 31/55; A61K 31/5377; A61K 31/454; A61K 31/40; A61K 31/397

(52) U.S. Cl. 514/18; 514/19; 514/210.16; 514/217.11; 514/317; 514/423; 514/478; 514/275; 514/235.2; 514/253.13; 514/254.01

(57) ABSTRACT

The instant invention is drawn to methods useful for the treatment or the prevention of a viral infection. The methods include administering at least one compound that is an inhibitor of cathepsin L to an individual. The methods are particularly useful in individuals infected with, or at risk of infection with, SARS virus or Ebola virus. The invention also includes methods of identifying potential therapeutics for use in the methods of treatment or prevention of a viral infection.

### CROSS REFERENCE TO RELATED APPLICATIONS

This application claims the benefit pursuant to 35 U.S.C. §119(e) to U.S. provisional patent application 60/693,028, which was filed on Jun. 22, 2005 and which is incorporated herein by reference in its entirety.

### STATEMENT REGARDING FEDERALLY SPONSORED RESEARCH OR DEVELOPMENT

This invention was supported in part by funds obtained from the U.S. Government, (NIH NIAID U54 A1057168, NIH RO1 A143455 and R21 A1059172) and the U.S. Government may therefore have certain rights in the invention.

### BACKGROUND OF THE INVENTION

The sudden appearance of infectious, deadly human diseases have been a recurrent scourge throughout mankind's history. In recent decades, the development of techniques to identify the etiology of a disease, as well as the discovery of antibacterial medications, e.g. antibiotics, and the development of vaccines, have reduced the danger of a large number of such diseases. Yet such diseases continue to appear, with sometimes devastating loss of life. Each such disease requires research into its etiology and research to discover prophylactics and therapeutics.

In recent years, two such infectious diseases have emerged: severe acute respiratory syndrome (SARS) and Ebola. The viral etiology for each of these diseases has been identified. SARS is an acute respiratory illness caused by a newly described coronavirus (SARS-CoV) (Rota et al., 2003, Science 300:1394-1399), the result of a zoonosis of a highly related animal coronavirus (Guan et al., 2003, Science 302:276-278). Mortality rates are estimated at approximately 10% with significantly increased mortality associated with advanced age (Poutanen et al., 2003, N Engl J Med 348:1995-2005). Ebola virus (EboV), which causes a fatal hemorrhagic fever in humans, is a filovirus and also appears to be zoonotic. Ebola virus infection is typically highly lethal. In some instances of Ebola outbreaks, mortality has been as high as 90% (www(dot)cdc(dot)gov/ncidod/dvrd/spb/mnpages/dispages/filoviruses(dot)htm). While an Ebola vaccine has recently been shown to be effective in monkeys (Jones et al., 2005, Nat Med Epub 5 Jun. 2005), there are currently no licensed vaccines available for either virus. Furthermore, no therapeutics have been identified to date for either disease.

Numerous studies have suggested potential vaccines or therapeutic treatments for SARS-CoV infection. S protein can provoke strong neutralizing antibody responses, and passive transfer of antibody is protective in animal models (Traggiai et al., 2004, Nat Med. 10(8): 871-5). These findings suggest that an effective vaccine could offer protection. Using a novel pseudotype assay system (Simmons et al., 2004, PNAS 101(12): 4240-5), it has recently shown that SARS-CoV infected individuals rapidly and strongly develop long-lived neutralizing antibodies (Temperton, 2005, Emerg Infect Dis. 11(3): 411-6), suggesting that a vaccine able to stimulate similar levels of neutralizing antibodies may be able to help control spread with rapid, localized vaccination. Various killed virus and recombinant viral component vaccines have demonstrated stimulation of specific antibody and cytotoxic T cell induction, as well as protection, in animal models (Gao et al., 2003, Lancet 362(9399): 1895-6; Bisht et al., 2004, PNAS 101(17): 6641-6; Yang et al., 2004, J Virol 78(11): 5642-50; Zeng et al., 2004, Biochem Biophys Res Commun 315(4): 1134-9; Tang et al., 2004, DNA Cell Biol 23(6): 391-4). Development of a safe SARS-CoV protective vaccine is complicated, however, by the fact that the feline coronavirus, FIPV, shows distinct antibody-dependent enhancement of infection (Olsen et al., 1992, J Virol 66(2): 956-65). This raises the possibility that antibody induction will not only fail to protect in vivo, but might even be detrimental. While humanized neutralizing antibodies directed against S protein offer the potential to act both post-infection and as a prophylactic treatment (Sui et al., 2004, PNAS 101(8): 2536-41), antibody therapeutics are expensive to manufacture, store and administer. Moreover, SARS has yet to manifest itself as a recurring epidemic threat, such as influenza, making mass vaccination of populations an unlikely scenario.

General anti-viral drugs are also available that in many cases function by activating an anti-viral state within the host. While some clinical studies have suggested that ribavirin may have some positive effects on the clinical outcome of SARS, others have described no effect or even increased disease (reviewed in Zhaori, 2003, CMAJ 169(11):1165-6). Another common anti-viral compound, interferon, has shown more promising results in a preliminary clinical setting as well as in vitro and in animal models (reviewed in Cinatl et al., 2003, Lancet, 362(9380):293-4). Both compounds, however, can be associated with significant adverse effects, particularly anemia and renal dysfunction (Kurschel et al., 1991, Ren Fail 13(2-3):8793). This makes it very unlikely that such compounds would be considered for prophylactic use. While much has been learned about SARS-CoV since its discovery, there remains a great need to develop anti-viral drugs, particularly those capable of being utilized prophylactically in the event of another major outbreak. Since the enzymatic proteins of SARS-CoV are receiving much attention, the development of inhibitors to other steps of the viral life cycle will provide useful complementary inhibitors and increase the likelihood of the development of drugs with low toxicity capable of being administered prophylactically.

Several stages in the SARS-CoV life cycle represent attractive targets for potential anti-SARS-CoV therapeutics. These include, target cell binding, entry and targets within the viral replicase machinery such as polymerase and viral protease activities. A zinc metalloproteinase, angiotensin-converting enzyme 2 (ACE2) has recently identified as a receptor for SARS-CoV (Li et al., 2003, Nature 426(6965):450-4; Wang et al., 2004, Biochem Biophys Res Commun 315(2):439-44). Inhibition of target cell binding and inhibition of entry are attractive as targets for inhibitors as inhibition at either of these stages prevents the initiation of any steps towards viral replication. The enzymatic processes of a virus are often good targets for anti-viral drugs due to their specialized mode of action. Much effort is being put into screens for useful compounds, as well as rational drug design, with some pre-existing drugs appearing to be effective in vitro (Yamamoto et al., 2004, Biochem Biophys Res Commun 318(3):719-25; Yang et al., 2003, PNAS 100(23):13190-5; Anand et al., 2003, Science 300(5626): 1763-7). However, many drugs targeted towards enzymatic processes are associated with significant side-effects due to cross-reactivity with host enzymes.

For the enveloped RNA viruses such as the filoviruses and the coronaviruses, distinct spikes of trimeric glycoproteins mediate the attachment, fusion and entry in target cells. A hallmark of these class I viral membrane fusion proteins is that they undergo a series of structural rearrangements that cause fusion between the viral and cellular membranes. The glycoproteins in the virion spikes are in an energetically unfavorable conformation, and an activating trigger, such as the low pH environment of an endosome or pH-independent interaction of the glycoproteins with a cellular receptor(s), is required to allow metastable protein complexes to refold into a more stable final form (reviewed in Skehel et al., 2000, Annu Rev Biochem 69:531-569). Viral glycoproteins are thus often classified as pH-dependent or pH-independent based upon the trigger required to activate their membrane fusion potential.

Class I fusion glycoproteins are synthesized as precursors that are often proteolytically processed into a surface subunit responsible for interacting with receptor and a membrane spanning subunit containing the machinery required to mediate membrane fusion. It is this proteolytic cleavage of a biosynthetic precursor that generates a metastable form of the protein and confers membrane fusion potential (Chen et al., 1998, Cell 95:409-417). For many viruses, an additional consequence of proteolytic processing is the generation of a highly hydrophobic fusion peptide at the amino terminus of the membrane spanning subunit. Insertion of this fusion peptide into the host cell membrane is a critical early step upon glycoprotein activation and precedes refolding of the glycoprotein into a stable six helix bundle that typifies membrane fusion for the class I glycoproteins.

The requirement for glycoprotein cleavage in coronavirus entry and membrane fusion is not well defined. Many coronaviruses, such as mouse hepatitis virus (MHV), contain a furin cleavage site within their spike (S) glycoproteins that yields S1 and S2 subunits (De Haan et al., 2004, J Virol 78:6048-6054; Frana et al., 1985, J Virol 56:912-920). Cleavage, however, is not absolutely required for infection, although lack of cleavage adversely affects S protein-mediated cell-cell fusion (De Haan et al., 2004, J Virol 78:6048-6054). Unlike MHV, however, SARS-CoV S protein does not appear to be efficiently proteolytically processed in cell culture (Krokhin et al., 2003, Mol Cell Proteomics 2:346-356; Simmons et al., 2004, PNAS 101:4240-4245; Song et al., 2004, J Virol 78:10328-10335). In this regard SARS-CoV more closely resembles other coronaviruses, such as feline infectious peritonitis virus (FIPV) (De Groot et al., 1989, Virology 171:493-502).

Entry into target cells mediated by retroviral pseudotypes containing SARS-CoV S protein is sensitive to lysosomotropic agents, such as ammonium chloride (Hofmann et al., 2004, J Virol 78:6134-6142; Nie et al., 2004, Biochem Biophys Res Commun 321:994-1000; Simmons et al., 2004, PNAS 101:4240-4245; Yang et al., 2004, J Virol 78:5642-5650). These findings suggest that SARS-CoV requires a low pH environment for infection. On the other hand, SARS-CoV S protein can mediate cell-to-cell fusion at neutral pH (Li et al., 2003, Nature 426:450-454; Simmons et al., 2004, PNAS 101:4240-4245), indicating that the triggers required to induce S protein-mediated fusion do not include an absolute requirement for an acidic pH. Given these discordant findings, it has been hypothesized that other factors sensitive to lysosomotropic agents, such as pH-dependent host endosomal proteins, may play a role in mediating SARS-CoV entry (Simmons et al., 2004, PNAS 101:4240-4245). Such a role has been demonstrated for cysteine proteases in the entry of EboV (Chandran et al., 2005, Science 308(5728):1643-5, Epub Apr. 14, 2005).

There is, therefore, an unmet need in the art for compositions and methods of inhibiting viral infection by SARS virus and Ebola virus. A safe and effective treatment of SARS patients, as well as safe protection of those potentially exposed or at high risk (e.g. hospital workers and emergency personnel), would be of significant benefit. The present invention meets these need.

### BRIEF SUMMARY OF THE INVENTION

The invention provides a method of treating a viral infection, particularly SARS or Ebola infection in a human in need of such treatment, comprising administering to the human at least one compound which is an inhibitor of cathepsin L.

The invention further provides a method of preventing a viral infection, particularly SARS or Ebola infection in a human likely to develop such a viral infection, comprising administering to the human at least one compound which is an inhibitor of cathepsin L.

Additionally, the invention provides methods of identifying potential therapeutic compounds for the treatment or prevention of SARS or Ebola viral infection in humans.

I. Compounds Useful in the Invention

A. Compounds Containing a Valine-Phenylalanine Moiety.

According to a first embodiment of the invention, certain compounds containing a Val-Phe structure act to inhibit cathepsin L and are thereby useful in the method of the invention. Examples of such compounds include Z-Val-Phe-CHO, Z-Val-Phe-FMK, Boc-Val-Phe-4-chlorobenzyl, Z-Val-Phe-NHO-enzyl, Z-Val-Phe-NHO-4-methoxybenzyl, and Z-Val-Phe-NHO-4-methylbenzyl.

B. Disulfide Compounds.

According to a second embodiment of the invention, disulfide compounds according to Formula I act to inhibit cathepsin L and are thereby useful in the method of the invention. Formula I is defined as:

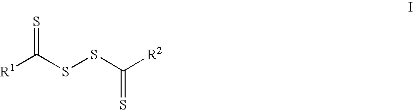

wherein R¹ and R² are independently selected from the group consisting of (C₃-C₁₀)hydrocarbyl, preferably —(C₃-C₁₀)cycloalkyl, —(C₅-C₁₀)cycloalkenyl, or aryl, more preferably —(C₃-C₈)cycloalkyl, cyclopentadienyl, or phenyl; and —N((C₁-C₆)alkyl)₂, wherein —N((C₁-C₆)alkyl)₂ includes moieties wherein the two alkyl groups combine to form a saturated heterocycle containing one nitrogen atom and from 4 to 7 carbon atoms.

According to one sub-embodiment of compounds according to Formula I, R¹ and R² are identical.

More preferably, R¹ and R² are both selected from the group consisting of —N(Et)₂, cyclohexyl, cyclopentyl, cycloheptyl, phenyl, cyclopentadienyl, cyclobutyl and cyclopropyl.

Most preferably, the thiuram disulfide compound is tetraethylthioperoxy-dicarbonic diamide.

C. Cyclopropenone Derivatives.

According to a third embodiment of the invention, cyclopropenone compounds according to Formula II act to inhibit cathepsin L and are thereby useful in the method of the invention. Formula II is defined as:

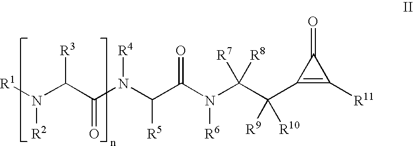

wherein:

R¹ is —H, R¹²—CO—, R¹²—NH—CO— or R¹²—SO₂—; wherein R¹² is C₁-C₂₀ alkyl optionally substituted by one or more substituents selected from the group consisting of C₃-C₁₅ cycloalkyl, optionally substituted C₆-C₁₄ aryl, C₃-C₁₅ cycloalkyloxy, optionally substituted C₆-C₁₄ aryloxy, optionally substituted C₆-C₁₄ arylthio, optionally substituted C₆-C₁₄ arylsulfonyl, optionally substituted C₇-C₂₀ aralkyloxy, optionally substituted heterocyclic, oxo, hydroxyl, C₁-C₁₀ alkoxycarbonyl group and carboxyl group; C₃-C₁₅ cycloalkyl; optionally substituted C₆-C₁₄ aryl or optionally substituted heterocyclic);

R², R⁴ and R⁶ each are independently —H or C₁-C₁₀ alkyl optionally substituted by C₁-C₅ alkoxy group or C₁-C₅ alkylthio group;

R₃, R₅ and R₇ each are independently —H, C₁-C₂₀ alkyl optionally substituted by C₃-C₁₀ cycloalkyl, C₃-C₁₀ cycloalkyl or optionally substituted C₇-C₂₀ aralkyl;

R⁸ is —H or C₁-C₂₀ alkyl;

R₇ and R₆ taken together may form C₃-C₁₅ cycloalkyl group;

R⁹ is hydroxyl group or C₂-C₁₀ acyloxy;

R¹⁰ is —H;

R⁹ and R¹⁰ taken together may form oxo;

R¹¹ is —H, C₁-C₂₀ alkyl group optionally substituted by C₃-C₁₅ cycloalkyl, C₃-C₁₅ cycloalkyl, C₂-C₂₀ alkenyl group, optionally substituted C₆-C₁₄ aryl group, optionally substituted C₇-C₂₀ aralkyl group, optionally substituted heterocyclic group or —C(R¹³)(R¹⁴)—OH; wherein R¹³ and R¹⁴ each are independently —H, C₁-C₂₀ alkyl, optionally substituted C₇-C₂₀ aralkyl or optionally substituted C₆-C₁₄ aryl;

R¹³ and R¹⁴ taken together may form C₃-C₁₅ cycloalkyl group; and

n is 0 or 1, or pharmaceutically acceptable salts thereof.

Compounds according to Formula II are disclosed in U.S. Pat. No. 5,328,909, the entire disclosure of which is incorporated herein.

D. 1-Oxytriazole and 1-oxyimidazole derivatives.

According to a fourth embodiment of the invention, 1-oxytriazole and 1-oxyimidazole compounds according to Formula III act to inhibit cathepsin L, and are thereby useful in the method of the invention. Formula III is defined as:

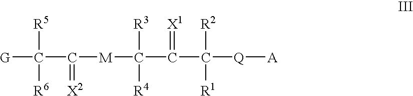

wherein:

M is O, NR⁷ or CR¹R², preferably NR⁷;

X¹ is O, S or NR⁷, preferably O;

X² is O, S, NR⁷ or two H atoms, preferably O;

Q is O, S or NR¹, preferably O;

R¹ and R² are independently —H, —(C₁-C₁₀)alkyl, —(C₁-C₁₀)heteroaryl, —(C₁-C₁₀)alkanoyl, or aroyl, wherein the alkyl, heteroaryl, alkanoyl and aroyl groups are optionally substituted with J;

R³, R⁴, R⁵ and R⁶ are independently —H, (C₁-C₁₀)alkyl, aryl, or heteroaryl, wherein the alkyl, aryl and heteroaryl groups are optionally substituted with J;

Preferably, R¹, R² and R⁴ are —H; and R³ is —H, n-butyl, isobutyl or benzyl;

R⁷ and R⁸ are independently —H, (C₁-C₁₀)alkyl, aryl, or heteroaryl, wherein the alkyl, aryl and heteroaryl groups are optionally substituted with J;

J is halogen, —COOR⁷, R⁷OCO—, R⁷OCONH—, —OH, —CN, —NO₂, —NR⁷R⁸, —N═C(R⁷)R⁸, —N═C(NR⁷R⁸)₂, —SR⁷, —OR⁷, phenyl, naphthyl, heteroaryl, or —(C₃-C₈)cycloalkyl;

G is —NH₂, —NHR¹, —CH₂R¹, —CH₂C(O)B, carbobenzyloxy-NH—, succinyl-NH—, R⁷O-succinyl-NH—, R⁷OC(O)NH—, —CH₂C(O)-(xanthen-9-yl), or —CH₂COR⁹;

wherein R⁹ is an alkyl, aryl, or arylalkyl group containing up to 13 carbons; or AA¹NHC(O)OCH₂ C₆ H₅ wherein AA¹ is one of the 20 natural amino acids or its opposite antipode;

B is —(C₁-C₁₀)alkyl, —(C₁-C₁₀)aralkyl, aryl having 1 to 3 carbocyclic rings, or heteroaryl having 1 to 3 rings, wherein the alkyl, aralkyl, aryl and heteroaryl groups are optionally substituted with J; and

A has the structure:

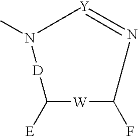

wherein: Y is N or CR¹;

W is a double bond or a single bond;

D is C═O or a single bond;

E and F are independently R¹, R², J; or when taken together E and F comprise an aliphatic carbocyclic ring having from 5 to 7 carbons, an aromatic carbocyclic ring having from 5 to 7 carbons, an aliphatic heterocyclic ring having from 5 to 7 atoms, or an aromatic heterocyclic ring having from 5 to 7 atoms;

wherein the aliphatic heterocyclic ring and the aromatic heterocyclic ring each have from 1 to 4 heteroatoms; and the aliphatic carbocyclic ring, the aromatic carbocyclic ring, the aliphatic heterocyclic ring, and the aromatic heterocyclic ring are each optionally substituted with J.

Compounds according to Formula III are disclosed in U.S. Pat. No. 5,498,616, the entire disclosure of which is incorporated herein.

E. Ketone Derivatives.

According to a fifth embodiment of the invention, ketone compounds according to Formula IV act to inhibit cathepsin L and are thereby useful in the method of the invention. Formula IV is defined as:

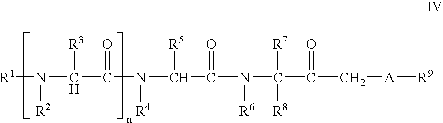

wherein:

R¹ is —H, R¹⁰—CO—, R¹⁰—O—CO—, R¹⁰—SO₂— or R₁₀—NH—CO—; wherein R₁₀ is C₁-C₂₀ alkyl optionally substituted by one or more substituents selected from the group consisting of C₃-C₁₅ cycloalkyl, C₃-C₁₅ cycloalkenyl, optionally substituted C₆-C₁₄ aryl, optionally substituted and partially hydrogenated C₁₀-C₁₄ aryl, fluorenyl, optionally substituted heterocyclic, C₃-C₁₅ cycloalkyloxy, optionally substituted C₆-C₁₄ aryloxy, optionally substituted and partially hydrogenated C₆-C₁₄ aryloxy, optionally substituted heterocyclic oxy, optionally substituted C₇-C₂₀ aralkyloxy and optionally substituted C₆-C₄ arylthio; C₃-C₁₅ cycloalkyl; optionally substituted C₆-C₁₄ aryl; optionally substituted and partially hydrogenated C₆-C₁₄ aryl; optionally substituted C₂-C₁₀ alkenyl and optionally substituted heterocyclic;

R², R⁴ and R⁶ each are independently —H or C₁-C₅ alkyl;

R³ and R⁵ each are independently —H, C₇-C₂₀ aralkyloxy, optionally substituted C₆-C₁₄ aryl, C₁-C₁₀ alkoxy or optionally substituted C₁-C₂₀ alkyl; or

R² and R³ and/or R⁴ and R⁵ taken together may form an optionally substituted nitrogen-containing heterocyclic ring;

R⁷ is C₁-C₂₀ alkyl optionally substituted by one or more substituents selected from the group consisting of C₃-C₁₅ cycloalkyl, hydroxyl, C₁-C₅ alkoxy optionally substituted by heterocyclic, C₆-C₁₄ aryloxy, C₇-C₂₀ aralkyloxy, C₁-C₅ alkylthio optionally substituted by heterocyclic, C₆-C₁₄ arylthio, C₇-C₂₀ aralkylthio, carboxyl, carbamoyl, C₂-C₆ alkoxycarbonyl, heterocyclic and optionally substituted C₆-C₁₄ aryl; —H; C₇-C₂₀ aralkyloxy; optionally substituted C₆-C₁₄ aryl and C₁-C₁₀ alkoxy;

R⁸ is —H, C₁-C₅ alkyl or optionally substituted C₇-C₂₀ aralkyl; or

R⁷ and R⁸ taken together may form optionally substituted benzylidene group or C₃-C₁₅ cycloalkyl;

A is —S—, —SO—, —SO₂—, —O— or —N(R¹¹)—; wherein R¹¹ is —H or optionally substituted C₁-C₂₀ alkyl), and

(1) when A is —S—, —SO— or —SO₂—, then R⁹ is optionally substituted C₆-C₁₄ aryl group or —(CH₂)ₘ—X; wherein X is —H, —OH, C₁-C₅ alkylthio, C₂-C₆ alkoxycarbonylamino, optionally substituted heterocyclic, —NH₂, C₁-C₅ monoalkylamino, C₂-C₁₀ dialkylamino, C₂-C₆ acylamino, halogen, C₁-C₅ alkoxy, optionally substituted C₆-C₁₄ aryl or optionally substituted C₆-C₁₄ aryloxy; and m is 0 or an integer of 1 to 15; provided that if R¹ is benzyloxycarbonyl, R⁴, R⁶ and R⁸ all are —H, R⁵ is benzyl, R⁷ is methyl and n is 0, then -A-R⁹ is not methylthio;

(2) when A is —O—, then R⁹ is —H or —(CH₂)₁—X (in which 1 is an integer of 1 to 15; and X is as defined above); or

(3) when A is —N(R¹¹)—, then R⁹ is optionally substituted C₆-C₁₄ aryl or —(CH₂)ₘ—X (in which X and m are as defined above); or R⁹ and R¹¹ taken together may form an optionally substituted nitrogen-containing heterocyclic ring; and

n is 0 or 1, or pharmaceutically acceptable salts thereof.

According to one preferred embodiment of compounds according to Formula IV;

R¹ is R¹⁰—CO—; wherein R¹⁰ is chromon-2-yl;

R², R⁴ and R⁶ each are independently —H or C₁-C₅ alkyl;

R³ and R⁵ each are independently C₁-C₆ alkyl;

R⁷ is C₁-C₆ alkyl group optionally substituted by a phenyl group;

R⁸ is —H or C₁-C₅ alkyl;

A is —S—, —SO— or —SO₂—;

R⁹ is —(CH₂)ₘ—X in which X is furanyl; and m is 0 or an integer of 1 to 15; and n is 0; or a pharmaceutically acceptable salt thereof.

Compounds according to Formula IV are disclosed in U.S. Pat. No. 5,639,783, the entire disclosure of which is incorporated herein.

F. Aminoketone Derivatives.

According to a sixth embodiment of the invention, aminoketone compounds according to Formula V act to inhibit cathepsin L, and are thereby useful in the method of the invention. Formula V is defined as:

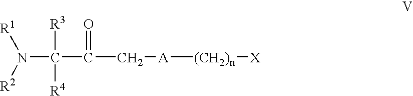

wherein:

R¹ is hydrogen,

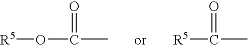

R⁵ is selected from the group consisting of C₁-C₂₀ alkyl, optionally substituted by one or more substituents selected from the group consisting of C₆-C₁₄ aryl optionally substituted by one or more substituents, fluorenyl, a heterocyclic residue optionally substituted by one or more substituents, C₃-C₁₅ cycloalkyl, C₃-C₁₅ cycloalkyloxy, C₆-C₁₄ aryloxy optionally substituted by one or more substituents, C₇-C₂₀ aralkyloxy optionally substituted by one or more substituents, C₆-C₁₄ arylthio optionally substituted by one or more substituents, —OH, C₂-C₁₀ acyloxy, C₂-C₁₀ alkenyl optionally substituted by C₆-C₁₄ aryl optionally substituted by one or more substituents, and a heterocyclic residue optionally substituted by one or more substituents,

R² and R⁴ are independently —H or C₁-C₅ alkyl,

R³ is —H, C₁-C₂₀ alkyl optionally substituted by one or more substituents, or C₆-C₁₄ aryl optionally substituted by one or more substituents, or R³ and R⁴ taken together, form a C₁-C₁₀ alkylene,

-A- is an oxygen atom, a sulfur atom or

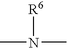

R⁶ is —H or C₁-C₅ alkyl;

n is an integer of from 1 to 10, and

X is a heterocyclic residue optionally substituted by one or more substituents.

Compounds according to Formula V are disclosed in U.S. Pat. No. 5,424,325, the entire disclosure of which is incorporated herein.

G. α-Aminoketone Derivatives.

According to an seventh embodiment of the invention, α-aminoketone compounds according to Formula VI, and pharmaceutically acceptable salts thereof, act to inhibit cathepsin L, and are thereby useful in the method of the invention. Formula VI is defined as:

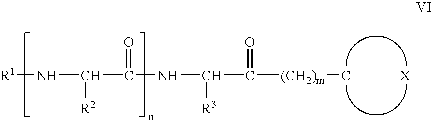

wherein:

R¹ is —H,

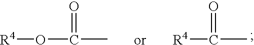

R⁴ is C₁-C₂₀ alkyl optionally substituted by one or more substituents selected from the group consisting of C₃-C₁₅ cycloalkyl, C₆-C₁₄ aryl optionally substituted by one or more substituents, a heterocyclic residue optionally substituted by one or more substituents, C₃-C₁₅ cycloalkyloxy, C₆-C₁₄ aryloxy optionally substituted by one or more substituents, C₇-C₂₀ aralkyloxy optionally substituted by one or more substituents, C₆-C₁₄ arylthio optionally substituted by one or more substituents, C₂-C₁₀ alkenyl optionally substituted by C₆-C₁₄ aryl optionally substituted by one or more substituents; C₆-C₁₄ aryl optionally substituted by one or more substituents and a heterocyclic residue optionally substituted by one or more substituents;

R² and R³ are independently —H or C₁-C₂₀ alkyl optionally substituted by one or more substituents;

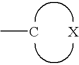

is a heterocyclic group containing at least one heteroatom selected from the group consisting of N, S and O, which heterocyclic group is optionally substituted by one or more substituents selected from the group consisting of halogen and C₁-C₃ alkyl;

n is 0 or 1 and m is an integer from 1 to 5.

Compounds according to Formula VI are disclosed in U.S. Pat. No. 5,422,359, the entire disclosure of which is incorporated herein.

H. α-Amino Fluoroketone Derivatives.

According to a eighth embodiment of the invention, α-amino fluoroketone compounds according to Formulae VIia, VIIb, VIIc and VIId, and pharmaceutically acceptable salts thereof, act to inhibit cathepsin L and are thereby useful in the method of the invention. Formulae VIIa, VIIb, VIIc and VIId are defined as:

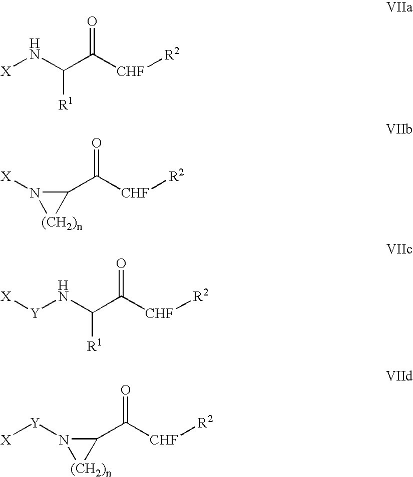

wherein:

R¹ and R² are independently selected from the group consisting of —H, optionally substituted —C₁-C₆ alkyl; aryl and aryl(C₁-C₄)alkyl;

n is an integer from 1-4 inclusive;

X is a peptide end-blocking group; and

Y is an amino acid or peptide chain of from 1-6 amino acids.

Compounds according to Formulae VIIa, VIIb, VIIc and VIId are disclosed in U.S. Pat. No. 4,518,528, the entire disclosure of which is incorporated herein.

I. Lactol Derivatives.

According to a ninth embodiment of the invention, lactol compounds according to Formula VIIIa and VIIIb, and pharmaceutically acceptable salts thereof, act to inhibit cathepsin L and are thereby useful in the method of the invention. Formulae VIIIa, is defined as:

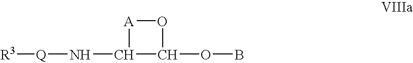

wherein:

Q is one or two optionally substituted amino acid residues;

R³ is an optionally esterified carboxyl group or an acyl group;

A is an alkylene group;

B is —H, an optionally substituted alkyl group or an acyl group,

or a salt of such a compound, and

Formulae VIIIb, is defined as:

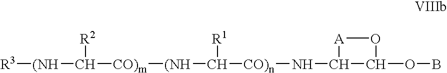

wherein:

R¹ and R² may be the same or different and each is —H or an optionally substituted hydrocarbon group;

R³ is an optionally esterified carboxyl group or an acyl group;

A is an alkylene group;

B is —H, an optionally substituted alkyl group or an acyl group;

m and n are each independently 0 or 1;

provided that where both m and n are 0, R³ is an optionally esterified carboxyl group or an acyl group having not less than 7 carbon atoms, or a salt of such a compound.

Compounds according to Formnulae VIIIa and VIIIb are disclosed in U.S. Pat. No. 5,496,834, the entire disclosure of which is incorporated herein.

J. L-Tryptophanyl Derivatives.

According to an tenth embodiment of the invention, L-tryptophanyl compounds according to Formula IX, and pharmaceutically acceptable salts thereof, act to inhibit cathepsin L and are thereby useful in the method of the invention. Formula IX, is defined as:

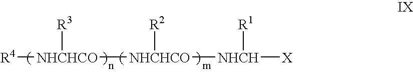

wherein:

R¹ is —H or an optionally substituted arylalkyl, heterocyclic-alkyl or lower alkyl group;

R² and R³, independently are —H or a hydrocarbon residue which may be substituted;

R⁴ is an optionally substituted alkanoyl, sulfonyl, carbonyloxy, carbamoyl or thiocarbamoyl group;

X is —CHO or —CH₂OB, wherein B is —H or a protecting group of hydroxyl group; and

m and n independently are 0 or 1;

provided that R⁴ is an alkanoyl group substituted by aryl, a sulfonyl group substituted by aryl having more than 9 carbon atoms or by lower alkyl or a carbamoyl or thiocarbamoyl group which may be substituted when R¹ is an unsubstituted lower alkyl, arylalkyl or methylthiomethyl group, R² and R³ independently are a lower alkyl or arylalkyl group, X is —CHO, m is 1 and n is 0 or 1, or a salt thereof.

Compounds according to Formula IX are disclosed in U.S. Pat. Nos. 5,498,728 and 5,639,781, the entire disclosures of which are incorporated herein.

K. Sulfonamide Derivatives.

According to an eleventh embodiment of the invention, sulfonamide compounds according to Formula X, and pharmaceutically acceptable salts thereof, act to inhibit cathepsin L and are thereby useful in the method of the invention. Formula X, is defined as:

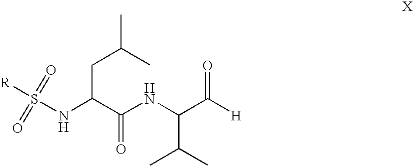

wherein:

R is C₆₋₁₄ aryl (phenyl, naphthyl, anthryl, etc.) which may have one or more substituents selected from a group consisting of a halogen such as fluorine, chlorine, bromine; C₁₋₅ alkyl such as methyl, ethyl, propyl, iso-propyl, butyl, iso-butyl, sec-butyl, tert-butyl, pentyl, isopentyl, neopentyl; trifluoromethyl; C₁₋₅ alkoxy such as methoxy, ethoxy, propoxy, iso-propoxy, butoxy, iso-butoxy, tert-butoxy, pentyloxy, iso-pentyloxy; C₁₋₅ cyclic acetal residue such as methylenedioxy, ethylenedioxy, propylenedioxy, butylenedioxy; hydroxyl; C₂₋₆ acyloxy such as acetoxy, propionyloxy, butyryloxy, valeryloxy; formyl; carboxyl; C₂₋₆ alkoxycarbonyl such as methoxycarbonyl, ethoxycarbonyl, propoxycarbonyl, isopropoxycarbonyl, butoxycarbonyl, isobutoxycarbonyl, tert-butoxycarbonyl, pentyloxycarbonyl; oxo; C₂₋₆ acyl such as acetyl, propionyl, butyryl, valeryl; amino; C₁₋₅ monoalkylamino such as methylamino, ethylamino, propylamino, isopropylamino, butylamino, isobutylamino, tert-butylamino, pentylamino, iso-pentylamino; C₂₋₁₀ dialkylamino such as dimethylamino, ethylmethylamino, diethylamino, methylpropylamino, diisopropylamino; C₂₋₆ acylamino such as acetylamino, propionylamino, isopropionylamino, butyrylamino, iso-butyrylamino, valerylamino; carbamoyl; and C₂₋₆ alkylcarbamoyl such as methylcarbamoyl, ethylcarbamoyl, propylcarbamoyl, isopropylcarbamoyl, butylcarbamoyl, tert-butylcarbamoyl, pentylcarbamoyl (hereinafter referred to as ‘Group 1’) or a heterocyclic residue having 1 to 4 hetero atoms selected from a group consisting of oxygen, sulfur and nitrogen and having, in total, 5 to 10 carbon atoms constituting a ring, for example, furyl, pyranyl, benzofuranyl, iso-benzofuranyl, chromenyl, chromanyl, isochromanyl, thiophenyl, benzothiophenyl, pyrrolyl, pyrrolinyl, pyrrolidinyl, imidazolyl, imidazolinyl, imidazolydinyl, pyrazolyl, pyrazolinyl, pyrazolidinyl, triazolyl, tetrazolyl, pyridyl, 1-oxopyridyl, piperidinyl, pyrazinyl, piperazinyl, pyrimidinyl, pyridazinyl, indolizinyl, indolyl, indolinyl, iso-indolyl, isoindolinyl, indazolyl, benzimidazolyl, purinyl, quinolizinyl, quionolyl, iso-quinolyl, phthalazinyl, naphthyridinyl, quinoxalinyl, quinazolinyl, cinnolinyl, pteridinyl, oxazolyl, oxazolidinyl, isoxazolyl, isoxazolidinyl, thiazolyl, thiazolidinyl, isothiazolyl, isothiazolidinyl, dioxolanyl, dioxanyl, dithianyl, morpholinyl, thiomorpholinyl, which may have one or more substituents (substituents are selected from ‘Group 1’).

Compounds according to Formula X are disclosed in U.S. Pat. No. 5,506,243, the entire disclosure of which is incorporated herein.

L. Epoxysuccinic Acid Derivatives.

According to a twelfth embodiment of the invention, epoxysuccinic acid compounds according to Formula XI, and pharmaceutically acceptable salts thereof, act to inhibit cathepsin L and are thereby useful in the method of the invention. Formula XI, is defined as:

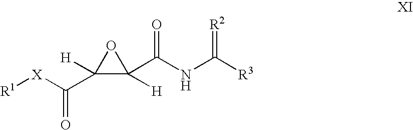

wherein:

R¹ is —H, a C₁-C₃₀ alkyl group, a C₆-C₄₀ aryl group, or a C₇-C₄₀ aralkyl group;

R² and R³ are independently a C₆-C₄₀ aryl group, a C₇-C₂₀ aralkyl group, or a C₃-C₁₀ alkyl group;

X is —O— or —NR⁴—; and R⁴ is —H, a C₁-C₁₀ alkyl group, or a C₇-C₂₀ aralkyl group.

Compounds according to Formula XI are disclosed in U.S. Pat. No. 5,843,992, the entire disclosure of which is incorporated herein.

M. Inhibitors Having a Heterocyclic Leaving Group.

According to a thirteenth embodiment of the invention, compounds according to Formula XII, having a heterocyclic leaving group, and pharmaceutically acceptable salts thereof, act to inhibit cathepsin L and are thereby useful in the method of the invention. Formula XII, is defined as:

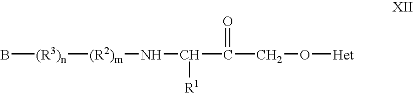

wherein:

B is —H or an amino acid blocking group for an N-terminal amino acid nitrogen;

R¹ is the amino acid side chain of the P₁ amino acid residue;

R² is the amino acid residue of the P₂ amino acid;

R³ is the amino acid residue of the P₃ amino acid;

n is 0 or 1;

m is 0 or 1; and

Het is the heterocyclic portion of the leaving group; wherein the heterocyclic leaving group includes a four-, five-, six- or seven-membered ring having at least one C and at least one of N, O or S in the ring. According to one preferred embodiment, Het is a furan. According to another preferred embodiment R² is a phenylalanyl residue. According to another preferred embodiment R¹ is a homophenylalanyl residue. According to another preferred embodiment n is 0 and m is 1.

As is conventional in the art, and as used above, amino acid residues may be designated as P₁, P₂, etc., wherein P₁ refers to the amino acid residue nearest the leaving group, P₂ refers to the amino acid residue next to P₁ and nearer the blocking group, etc. In dipeptide inhibitors therefore, P₂ is the amino acid residue nearest the blocking group.

Compounds according to Formula XII are disclosed in U.S. Pat. No. 5,663,380, the entire disclosure of which is incorporated herein.

N. Polypeptide Compounds.

According to a fourteenth embodiment of the invention, a polypeptide compound that acts as an inhibitor of cathepsin L useful in the method of the invention. One such polypeptide has an amino acid sequence represented by Seq. ID No. 1 in the appended sequence listing, is known to inhibit cathepsin L and is thereby useful in the method of the invention. The polypeptide of Seq. ID No. 1 is disclosed in U.S. Pat. No. 5,698,519, the entire disclosure of which is incorporated herein.

Other polypeptide compounds useful in the methods of the invention include neutralizing anti-cathepsin L antibodies.

O. Cysteine and Serine Protease Inhibitors Containing a Ketomethylene Group.

According to a fifteenth embodiment of the invention, compounds according to Formula XIII, having a ketomethylene group, and pharmaceutically acceptable salts thereof, act to inhibit cathepsin L and are thereby useful in the method of the invention. Formula XIII, is defined as:

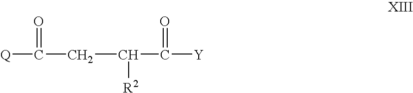

wherein:

Q is aryl having from about 6 to about 14 carbons, heteroaryl having from about 6 to about 14 ring atoms, aralkyl having from about 7 to about 15 carbons, alkyl having from 1 to about 10 carbons, said alkyl groups being optionally substituted with one or more J groups, heteroalkyl having from 2 to about 7 carbons, arylheteroalkyl wherein the aryl portion can be unfused or fused with the heteroalkyl ring, alkoxy having from 1 to about 10 carbons, aralkyloxy having from about 7 to about 15 carbons, a carbohydrate moiety optionally containing one or more alkylated hydroxyl groups, xanthene-9-yl, CH(i-C₄H₉)NHCbz, CH₂N(i-C₄H₉)Cbz, or Formula XIIIa or XIIIb:

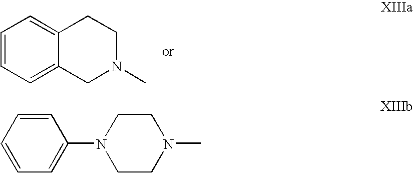

Y has the formula:

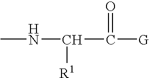

wherein R¹ and R² are independently —H, alkyl having from one to about 14 carbons, cycloalkyl having from 3 to about 10 carbons, or a natural or unnatural side chain of an L-amino acid, said alkyl and cycloalkyl groups being optionally substituted with one or more J groups;

J is halogen, lower alkyl, aryl, heteroaryl, amino optionally substituted with one to three aryl or lower alkyl groups, guanidino, alkoxycarbonyl, aralkoxycarbonyl, alkoxy, hydroxy, or carboxy; and

G is —H, —C(═O)NR³R⁴, —C(═O)OR³ or —CH₂R⁵;

wherein:

R³ and R⁴ are each independently —H, alkyl having from 1 to about 10 carbons, said alkyl groups being optionally substituted with one or more J groups, aryl having from about 6 to about 14 carbons, and aralkyl having from about 7 to about 15 carbons; and

R⁵ is halogen;

with the proviso that if G is —H and Q is alkyl substituted with J, and said J is an α-amino group, then the α-amino nitrogen must be tertiary (that is, trisubstituted with other than —H).

Compounds according to Formula XIII are disclosed in U.S. Pat. No. 5,827,877, the entire disclosure of which is incorporated herein.

P. Indole Nitrile Derivatives.

According to a sixteenth embodiment of the invention, compounds according to Formula XIV, having an indole nitrile group, and pharmaceutically acceptable salts thereof, act to inhibit cathepsin L and are thereby useful in the method of the invention. Formula XIV, is defined as:

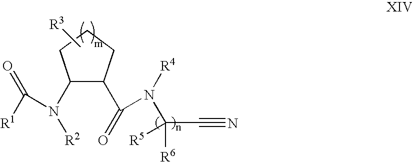

wherein:

m is 1, 2 or 3;

n is 1 or 2;

p is from 0 to 2;

R¹ is: optionally substituted indolyl; optionally substituted indazolyl; optionally substituted benzothiazole; optionally substituted indolizinyl; optionally substituted tetrahydropyridoindolyl; optionally substituted pyridinylthiophenyl; or optionally substituted benzopyrrplothiazolyl;

R², R³, R⁴ and R⁵ each independently is —H or alkyl; and

R⁶ is —H; alkyl, cycloalkyl, or —(CR^{a}R^{b})_{q}-A;

wherein R^{a} and R^{b} each independently are —H or alkyl; q is from 0 to 3; and

A is hydroxy, alkoxy, cyano, optionally substituted phenyl, optionally substituted pyridyl, optionally substituted imidazolyl, optionally substituted thienyl, —S(O)ᵣ—R^{c}, wherein r is from 0 to 2 and R^{c} is —H or alkyl, —COR^{d}, wherein R^{d} is hydroxy, alkoxy, morpholinyl, or cycloalkylamino; or —NR^{e}R^{f} wherein R^{e} and R^{f} each independently are —H or alkyl; or R^{e} and R^{f} together with the nitrogen to which they are attached may form a five or six membered ring that optionally includes an additional heteroatom selected from O, N and S;

and pharmaceutically acceptable salts, solvates or prodrugs thereof.

Compounds according to Formula XIV are disclosed in U.S. Pat. No. 6,759,428, the entire disclosure of which is incorporated herein.

For the above embodiments, the definitions of variables, e.g., R¹, R² etc., correspond only to the structure diagram with which they are associated in the text. Thus, for example, even though the symbol R¹ is employed in defining a structural element of a compound according to Formula I and a structural element of a compound according to Formula II, the definition of R¹ associated with Formula I is understood to correspond only to compounds of Formula I.

Q. Other Compounds Known to Inhibit Cathepsin L

According to a seventeenth embodiment of the invention compounds selected from the group consisting of N—[N-(L-3-trans-carboxyoxirane-2-carbonyl)-L-leucyl]-agmatine, Leupeptin, Z-Phe-Gly-NHO-Bz, Z-Phe-GIy-NHO-Bz-pOMe, Z-Phe-Gly-NHO-Bz-pMe, Z-Phe-Phe-FMK, Z-Phe-Tyr-CHO, Z-Phe-Tyr(t-Bu)-diazomethylketone, 1-Napthalenesulfonyl-Ile-Trp-CHO, Z-Phe-Tyr(OtBu)-COCHO, Z-Ill-FMK, Pindobind, L-3-carboxy-trans-2,3-epoxypropyl-leucylamide-(3-guanizino)butane (Ep-475), 1-(6-((8R,9S,13S,14S)-7,8,9,11,12,13,14,15,16,17-decahydro-3-methoxy-13-methyl-6H-cyclopenta[a]phenanthren-17-ylamino)hexyl)-1H-pyrrole-2,5-dione and 3,5 dinitrocatechol act to inhibit cathepsin L, and are thereby useful in the method of the invention.

### BRIEF DESCRIPTION OF THE DRAWINGS

For the purposes of illustrating the invention, there are depicted in the drawings certain embodiments of the invention. However, the invention is not limited to the precise arrangements and instrumentalities of the embodiments depicted in the drawings.

FIGS. 1A-1D are a series of graphs and images as follows depicting the effect of trypsin treatment of SARS-CoV S protein mediated infection.

FIG. 1A is a graph of luciferase activity in 293T and Vero E6 cells infected with HIV(SAR S) pseudotype virions. HIV(SARS S) pseudotype virions were treated in solution with TPCK-trypsin for 10 minutes and then used to infect 292T cells or Vero E6 cells. Luciferase was assayed after 48 hours.

FIG. 1B is a graph of luciferase activity in 293T cells infected with HIV(SAR S), HIV(SAR S(K672L)) or HIV(SAR S(R667A)) pseudotype virions. Trypsin pre-treatment of S protein and mutants inactivates infectivity for wild-type and S(K672L), but not S(R667A). In FIGS. 1B and 1C, pseudotype infection of 293T cells transiently expressing human ACE2 was assessed as luciferase activity presented as a percentage of no trypsin controls (approximately 40,000 relative light units (RLU)). Results represent the means of samples run in triplicate (±SD). Similar results were seen in two subsequent assays.

FIG. 1C is a bar graph depicting that trypsin treatment bypasses anrnonium chloride inhibition. Pseudotypes were bound to mock or ammonium chloride (20 mM) treated 293T cells transiently expressing human ACE2. Cells were then incubated with either PBS or TPCK-trypsin (15 pg/ml). Results are presented as a percentage of no ammonium chloride, no trypsin controls (approximately 4000 and 10,000 RLU for HIV-luc(SARS S) and HIV-luc(VSV-G) respectively), and represent the means of samples run in triplicate (±SD).

FIG. 1D is a series of images of cells immunostained fro S protein expression. Trypsin treatment bypasses ammonium chloride inhibition of live virus. Mock (middle panels) or 25 mM ammonium chloride (far right panels) pre-treated Vero E6 cells were spin infected with live, replication-competent SARS-CoV at a MOI of approximately 0.5 and then incubated with either DMEM alone (top panels) or DMEM containing 15 μg/ml TPCK-trypsin (bottom panels). Infection was allowed to progress for 48 hours, before immunostaining for S protein expression.

FIG. 2A are images of blots depicting trypsin-mediated cleavage of S protein. Trypsin specifically digests S protein at Arg667. Concentrated pseudovirions were mock or trypsin (10 μg/ml) treated for 10 minutes at 25° C., lysed and analyzed by SDS PAGE. Protein markers (M), starting from the top, are 220, 120, 100 and 80 kDa. Blots were probed with antiserum to the C-terminal third of the extracellular domain of S protein (left panel) or with a monoclonal antibody to the extreme N-terminus of S protein (right panel).

FIG. 2B is a bar graph depicting the effect of Arg667 on trypsin-mediate cleavage of S protein. Arg667 is not involved in trypsin-mediated bypass of ammonium chloride inhibition. Results are presented as a percentage of no ammonium chloride, no trypsin controls (approximately 25,000 and 10,000 RLU for HIV-luc(SARS S) and HIV-luc(R667A) respectively), and represent the means and of samples run in triplicate (±SD). Similar results were seen in a subsequent assay.

FIGS. 3A-3E are a series of graphs and images as follows depicting protease inhibitor sensitivity of pseudotyped viruses.

FIG. 3A is a graph depicting leupeptin, an inhibitor of serine and cysteine trypsin-like proteases, inhibition of S protein mediated infection. 293T cells were pre-incubated with leupeptin and then challenged with HIV-luc pseudovirions with different viral envelopes. Results are presented as a percentage of infection of untreated cells (approximately 3000 RLU for each envelope) and represent the means of samples run in triplicate (±SD). Similar results were seen in two subsequent assays.

FIG. 3B is a graph depicting leupeptin inhibition of replication-competent SARS-CoV infection of 293T cells transiently expressing ACE2. Cells were either pre-incubated with leupeptin for 1 hour and then exposed to virus for 3 hours in the continued presence of leupeptin (“Before & During Exposure”) followed by washing out of both virus and inhibitor, or exposed to virus for 3 hours, washed and then incubated for a further 4 hours with leupeptin (“After exposure”). Three days post-exposure, supernatant was harvested and cell-free levels of nucleoprotein were detected by ELISA. Results are expressed as optical density at 405 mn, following subtraction of no virus controls, and represent the means of samples run in triplicate (±SD).

FIG. 3C is a series of images of cells immunostained for S protein expression. Trypsin treatment bypasses leupeptin inhibition of live virus. Mock (middle panels) or 500 μg/ml leupeptin (far right panels) pre-treated Vero E6 cells were spin infected with live, replication-competent SARS-CoV at a MOI of approximately 0.5 and then incubated with either DMEM alone (top panels) or DMEM containing 15 μg/ml TPCK-trypsin (bottom panels). Infection was allowed to progress for 48 hours, before immunostaining for S protein expression.

FIG. 3D is a graph depicting the effect of E64c or Aprotinin on SAR-CoV S protein-mediated entry. A cysteine, but not a serine protease inhibitor, blocks SARS-CoV S protein-mediated entry. 293T cells transiently expressing ACE2 were pre-incubated with a cysteine protease inhibitor (E64c) or a serine protease inhibitor (Aprotinin) and then challenged with HIV-luc pseudovirions. Results are presented as a percentage of infection of untreated cells (approximately 1500 RLU for VSV-G and 6000 for SARS-CoV S protein) and represent the means of samples run in triplicate (±SD). Similar results were seen in two additional experiments.

FIG. 3E is a graph depicting the effect of Z-LLL-FMK or CA-074 on SAR-CoV S protein-mediated entry. An inhibitor of cathepsin B and L, but not a cathepsin B-specific inhibitor, blocks S protein mediated infection of Vero E6 cells. Vero E6 cells were pre-incubated with Z-LLL-FMK or CA-074 and then challenged with HIV-luc pseudovirions. Results are presented as a percentage of infection of untreated cells (approximately 15,000 RLU for VSV-G and 20,000 RLU for SARS-CoV S) and represent the means of samples run in triplicate (±SD). Similar results were seen on 293T cells and 293T cells transiently expressing ACE2.

FIG. 4 is a bar graph depicting ACE2-mediated infection of SARS-CoV S protein expressing cells. HIV-luc(ACE2) particles are infectious on S protein expressing cells. Pseudovirions incorporating ACE2 (termed HIV-luc(ACE2)), MLV amphotropic env, SARS-CoV S protein or no surface glycoprotein (termed HIV-luc(bald)) were used to infect 293T cells transfected with empty vector or plasmids encoding ACE2 or SARS-CoV S protein. Results are presented on a log₁₀ scale of relative light units and represent the mean of samples run in triplicate (±SD).

FIGS. 5A-5D are a series of graphs and schematics as follows.

FIG. 5A is a schematic of a model of intervirion fusion.

FIG. 5B is a bar graph depicting that intervirion fusion requires ACE2 and S protein. Target particles encoding luciferase were incubated with particles encoding GFP. Mixed virions were then used to infect HeLa/Tva cells that had been pre-treated with either medium or medium supplemented with leupeptin (20 μg/ml). Intervirion fusion was quantitated as luciferase activity 48 hours post infection. Results represent the means of samples run in triplicate (±SD).

FIG. 5C is a bar graph depicting the effect of trypsin cleavage on fusion. Trypsin cleavage promotes fusion mediated by wild type SARS S and S(R667A). Intervirion fusion between HIV-luc(ACE2) and either HIV-gfp(SARS S/ASLV-A) or HIV-gfp(SARS S(R667A)/ASLV-A) was quantified by luciferase activity 48 hours post infection of HeLa/Tva cells pre-treated with leupeptin (20 μg/ml). Results represent the means of samples run in triplicate (±SD).

FIG. 5D is a bar graph depicting that trypsin, but not low pH, enhances intervirion fusion. HIV-luc(ACE2) and HIV-GFP(SARS S/ASLV-A) particles were mixed and treated with TPCK-trypsin (10 μg/ml) for 10 minutes at 4° C. or pulsed at pH 5.0. Following further incubation in the presence of trypsin inhibitor or neutralization of acid conditions, mixed virus was used to infect HeLa/Tva cells pre-treated with leupeptin (20 μg/ml). Results represent the means of samples run in triplicate (±SD).

FIG. 6A-6C are bar graphs depicting cathepsin L mediated enhancement of S protein activation as follows.

FIG. 6A depicts data illustrating that cathepsin L enhances intervirion fusion. HIV-luc(ACE2) and HIV-GFP(SARS S/ASLV-A) particles were mixed and incubated for 10 minutes at 25° C. with preactivated 2 μg/ml cathepsin B (at pH 5.0), 2 μ.g/ml cathepsin L (at pH 6.0), cathepsin L buffer alone (at pH 6.0) or 10 μg/ml TPCK-trypsin (at pH 7.0). Mixed virus was then used to infect HeLa/Tva cells pre-treated with leupeptin (20 μg/ml). Results represent the means of samples run in quadruplicate (±SD). Similar results were observed in two subsequent experiments.

FIG. 6B is a bar graph depicting data illustrating that acidic conditions are required for cathepsin L mediated S protein activation. HIV-luc(ACE2) and HIV-GFP(SARS S/ASLV-A) particles were mixed and adjusted to various pH's. Following neutralization of acid conditions, mixed virus was used to infect HeLa/Tva cells pre-treated with leupeptin (20 μg/ml). Results represent the means of samples run in quadruplicate (±SD). Similar results were observed in a further experiment.

FIG. 6C is a bar graph depicting the effect of incubation temperature on trypsin cleavage. Following receptor interactions, an incubation step at physiological temperature is required prior to trypsin cleavage. HIV-luc(ACE2) and HIV-GFP(SARS S/ASLV-A) particles were mixed and incubated at 4° C. to allow binding. Samples were then either incubated at 37° C. for 15 minutes followed by a further 5 minute incubation at 4° C. or maintained at 4° C. throughout. TPCK-trypsin (10 pg/ml) digestion was then carried out at 4° C. for 15 minutes. Following neutralization in trypsin inhibitor, particles were incubated for a further 30 minutes at 37° C. to allow membrane fusion and then used to infect HeLa/Tva cells pre-treated with leupeptin (20 μg/ml). Results represent the means of samples run in quadruplicate (±SD). Similar results were observed in two further experiments.

FIGS. 7A-7C are a series of schematics of models as follows for class I viral glycoprotein cleavage and triggering.

FIG. 7A depicts a model of influenza hemagglutinin-mediated entry. For the majority of strains, HA is processed by secreted proteases in the extracellular matrix. Following attachment to sialic acid, virions are endocytosed and transported to acidic endosomes where the low pH environment triggers conformational rearrangements within HA and leading to membrane fusion.

FIG. 7B depicts a model of HIV envelope-mediated entry. Env is processed by furin-like proteases during transport to the cell surface. Mature, processed env is then incorporated into budding virions. Attachment to CD4 and coreceptor on the surface of target cells induces conformational rearrangements in env and membrane fusion.

FIG. 7C depicts a model of SARS-CoV S-mediated entry. S protein is not efficiently processed on mature virions. Binding to ACE2 on the surface of target cells induces initial conformational rearrangements within S protein to more efficiently expose protease cleavage sites. Following endocytosis, these cleavage sites are acted upon by pH-dependent endosomal proteases such as cathepsin L. Cleavage then allows completion of conformational rearrangements and induction of membrane fusion.

FIG. 8 is a graph depicting the results of a luciferase assay of pseudotype virion-infected 293T cells in the presence of cathepsin L inhibitor, MDL 28170. The IC50 for MDL 28170 determined using this pseudotype virion entry into cells is about 100 nM.

FIG. 9 is a graph depicting the effect of different concentrations of MDL 28170 on viral entry of four different pseudotyped virions into 292T cells. Y-axis is relative light units for luciferase. The data demonstrate that MDL 28170 (PN-001) inhibits entry of retroviral pseudotypes SARS CoV and EboV. VSV and MLV pseudotypes to not require cathepsin L activity for viral entry and therefore are not inhibited by PN-001.

FIGS. 10A-10E are graphs of cathepsin L inhibition curves for various compounds as follows. IC50 values are indicated in each graph. (10A) PN-001; (10B) PN-002; (10C) U73122; (10D) pindobind; and (10E) 3,5 dinitrocatechol. Data was obtained in 384-well plate assays using purified cathepsin L and a fluorogenic substrate.

FIGS. 11A-11D are a series of images as follows.

FIG. 11A is an image of a nanoliter microarray. Nanoliter volumes of glycerol were contact printed in a 384-microarray format at 400 spots/cm². The penny coin is present to indicate the scale of the microarray.

FIG. 11B is a series of images of aerosol deposition visualized under conditions of low carrier gas flow (<0.5 L/min). An aerosol of biological sample generated via an ultrasonic nozzle allowed metering of picoliter quantities of reactants into each individual glycerol reaction center.

FIG. 11C is a series of images of a nanoliter microarray at different times with respect to aerosol deposition. The array, images by differential interference microscopy, is shown before and immediately after aerosol deposition. The deposited aerosol rapidly dried within 7 seconds, leaving spots intact and maintaining individual reaction compartments; bar=200 micrometer.

FIG. 11D is an image of a nanoliter microarray. To demonstrate the uniformity of the aerosol deposition process and the absence of spot-to-spot intermixing, an aerosol of TRITC (1 nM in DMSO; yellow) was deposited at 400 nanoliters/sec for 4 seconds on an array initially containing alternating rows of AMC (1 mM; blue) and dye-free glycerol spots; bar=500 micrometer.

FIG. 12A is an image of a nanoliter microarray for screening the serine protease, thrombin. The 16×16 microarray contains thrombin, substrate boc-VPR-MCA and, in the central portion of the array, the thrombin inhibitor benzamidine. The concentrations of the inhibitor are indicated to the right of the microarray.

FIG. 12B is a bar graph depicting the dose response data for benzamidine inhibition of thrombin from the microarray in FIG. 12A.

FIGS. 13A-13D are a series of images and graphs as follows.

FIG. 13A is an image of a nanoliter microarray used for screening for inhibitors of caspase 6. Nanoliter screening of a compound library microarrayed at 1 mM in quadruplicate with human caspase 6 and a fluorogenic substrate, VEID-MCA, identified the location of an inhibitor compound (compound 1, C₁₆H₁1Br₃N₄O). The structure of compound 1 overlays the microarray.

FIGS. 13B and 13C are images of microarrays containing caspase 2 and caspase 4, respectively. When replicate microarrays were screened against caspase 2 (13B) or caspase 4 (13C), the identical location on the microarrays seen in FIG. 13A displayed low fluorescence emission indicative of enzyme inhibition.

FIG. 13D is a series of dose response curves compound 1 and caspases 2, 4 and 6. When tested in triplicate in a standard well-plate assay (2U/microliter caspase in 15 microliter reaction with 200 micromolar substrate in buffered saline at 22° C. and incubated 1200 seconds), compound 1 caused a dose-dependent inhibition of caspases 2, 4 and 6 with an IC50 of ˜0.5 to 5 millimolar against the three caspases, with 100% inhibition detected in all reactions at 10 millimolar. Compound 1 had no fluorescence emission or excitation overlap with the assay components.

FIG. 14 is an image of a nanoliter microarray used for screening for inhibitors of cathepsin L. Two regions of the array with hits for three compounds that inhibit cathepsin L cleavage are shown enlarged, along with the structures of the three compounds. Nanoliter screening of a test compound library (LOPAC) microarrayed in triplicate with human cathepsin L and a fluorogenic substrate, VEID-MCA, identified the location of several inhibitors, including MDL 28170, tetraethylthiuram disulfide and leupeptin. Not all hits are shown.

### DETAILED DESCRIPTION OF THE INVENTION

As the Examples herein demonstrate, SAR S CoV entry and EboV entry involve the cleavage activity of the endosomal pH-dependent protease, cathepsin L. As further demonstrated in the Examples, viral entry is reduced significantly in the presence of a cathepsin L inhibitor. The invention therefore provides methods of treating or preventing viral infection by administering to an individual in need at least one compound that is an inhibitor of cathepsin L. The invention further provides methods of identifying potential therapeutics for viral infection.

### Definitions

General

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Viral infection” as used herein refers to infection by a viral pathogen wherein there is clinical evidence of the infection based on symptoms or based on the demonstration of the presence of the viral pathogen in a biological sample from the individual.

As used herein an “individual” refers to an animal, preferably a mammal, including both non-human mammals and humans, and more preferably, refers to a human.

The expression “effective amount” when used to describe therapy to an individual suffering from a viral infection refers to the amount of a compound that results in a therapeutically useful effect on the symptoms of the viral infection.

“Treatment of a viral infection” as used herein encompasses alleviating, reducing the frequency of, or eliminating one or more symptoms of the infection.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks, directly or indirectly, the biological activity of the antigen.

As used herein, “viral pseudotype” refers to a virion containing the genome of one virus but the envelope proteins of both viruses. The production of viral pseudotypes has been used to study the glycoproteins of highly pathogenic viruses. Importantly, the glycoproteins on the pseudotype recapitulate the normal activity of these proteins in viral infection, including host range and tropism, receptor recognition, antibody neutralization and functional requirements. Advantageously, when designed appropriately, pseudotypes do not pose the health risk of the wild-type pathogenic viruses, thus making research with them considerably less risky.

A host cell that comprises a recombinant polynucleotide is referred to as a “recombinant host cell.” A gene, which is expressed in a recombinant host cell wherein the gene comprises a recombinant polynucleotide, produces a “recombinant polypeptide.”

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, DC, p. 574). Other examples of reporter genes include, but are not limited to, luciferase, green fluorescent protein (GFP), and β-glucuronidase.

As used herein, “PN-001” and MDL 28170 are alternative names for Z-Val-Phe-CHO, and are used herein interchangeably.

As used herein, “PN-002” and “tetraethylthiuram disulfide” are alternative names for “tetraethylthioperoxydicarbonic diamide”, and are used interchangeably herein.

As used herein, “U73122” refers to 1-(6-((8R,9S,13S,14S)-7,8,9,11,12,13,14,15,16,17-decahydro-3-methoxy-13-methyl-6H-cyclopenta[a]phenanthren-17-ylamino)hexyl)-1H-pyrrole-2,5-dione, and the names are used interchangeably herein.

As used herein, “Z-LLL-FMK” refers to Z-leu-leu-leu-fluoromethyl ketone.

Chemical

The term “alkyl”, by itself, or as part of another substituent, e.g., haloalkyl or aminoalkyl, means, unless otherwise stated, a saturated hydrocarbon radical having the number of carbon atoms designated (i.e. C₁-C₆ means the group contains one, two, three, four, five or six carbons) and includes straight, branched chain, cyclic and polycyclic groups. Examples include: methyl, ethyl, propyl, isopropyl, butyl, isobutyl, tert-butyl, pentyl, neopentyl, hexyl, cyclohexyl, norbomyl and cyclopropylmethyl. Preferred alkyl groups are —(C₁-C₆)alkyl. Most preferred is —(C₁-C₃)alkyl, particularly ethyl, methyl and isopropyl.

The expression “substituted alkyl” means alkyl, as defined above, substituted by one, two or three substituents preferably independently selected from the group consisting of halogen, —OH, —O(C₁-C₄)alkyl, —NH₂, —N(CH₃)₂, —CO₂H, —CO₂(C₁-C₄)alkyl, —CF₃, —CONH₂, —SO₂NH₂, —C(═NH)NH₂, —CN and —NO₂. Examples of substituted alkyls include, but are not limited to, 2,2-difluoropropyl, 2-carboxycyclopentyl and 3-chloropropyl.

The term “alkylene”, by itself or as part of another substituent means, unless otherwise stated, a divalent straight, branched or cyclic chain hydrocarbon radical having the designated number of carbons. The expression —C(═O)(C₁-C₄)alkylene-R includes one, two, three and four carbon alkylene groups. A substitution of a group such as R on alkylene may be at any substitutable carbon, i.e., the group, —C(═O)(C₄ alkylene)R, includes, for example (a), (b) and (c), in Scheme 1, below:

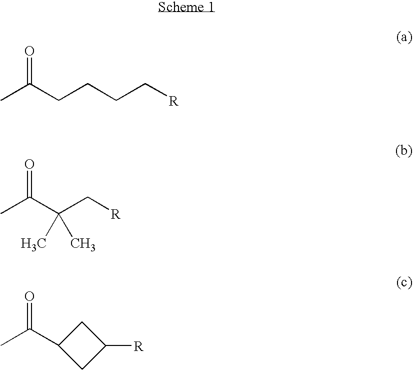

The term “amine” or “amino” refers to radicals of the general formula NRR′, wherein R and R′ are independently hydrogen or an optionally substituted hydrocarbyl radical, or wherein R and R′ combined form a heterocycle. Examples of amino groups include: —NH₂, methyl amino, diethyl amino, anilino, benzyl amino, piperidinyl, piperazinyl and indolinyl.

The term “aromatic” refers to a carbocycle or heterocycle having one or more polyunsaturated rings having aromatic character (4n+2) delocalized π (pi) electrons).

The term “aryl” employed alone or in combination with other terms, means, unless otherwise stated, a carbocyclic aromatic group containing one or more rings (typically one, two or three rings) wherein such rings may be attached together in a pendent manner, such as a biphenyl, or may be fused, such as naphthalene. Examples include phenyl, anthracyl and naphthyl. Preferred are phenyl and naphthyl, most preferred is phenyl.

The term “aryl-(Cₓ-C_{y})alkyl” or “arylalkyl” or “aralkyl” mean a radical wherein a carbon alkylene chain having the designated number of carbon atoms is attached to an aryl group, e.g., —CH₂CH₂-phenyl. Preferred is aryl(CH₂)— and aryl(CH(CH₃))—. The term “substituted aryl-(C₁-C₃)alkyl” means an aryl-(C₁-C₃)alkyl radical in which the aryl group is substituted. Preferred is substituted aryl(CH₂)—. Similarly, the term “heteroarylalkyl” means a radical wherein a carbon alkylene chain having the designated number of carbon atoms is attached to a heteroaryl group, e.g., —CH₂CH₂-pyridyl. Preferred is heteroaryl(CH₂)—. The term “substituted heteroaryl-(C₁-C₃)alkyl” means a heteroaryl-(C₁-C₃)alkyl radical in which the heteroaryl group is substituted. Preferred is substituted heteroaryl(CH₂)—.

The term “arylene,” by itself or as part of another substituent means, unless otherwise stated, a divalent aryl radical. Preferred are divalent phenyl radicals, particularly 1,4-divalent phenyl radicals.

The term “cycloalkyl” refers to ring-containing alkyl radicals. Examples include cyclohexyl, cyclopentyl, cyclopropyl methyl and norbornyl.

The term “hydrocarbyl” refers to any moiety comprising only hydrogen and carbon atoms. Preferred hydrocarbyl groups are (C₁-C₁₂)hydrocarbyl, more preferred are (C₁-C₈)hydrocarbyl, most preferred are benzyl and —(C₁-C₆)alkyl.

The term “hydrocarbylene” by itself or as part of another substituent means, unless otherwise stated, a divalent moiety comprising only hydrogen and carbon atoms. A substitution of another group —R on hydrocarbylene may be at any substitutable carbon, i.e., the expression —(C₁-C₆ hydrocarbylene)-R includes, for example, the structures shown in Scheme 2:

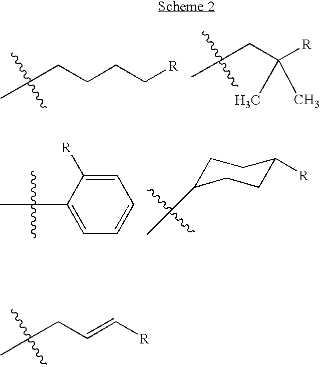

The term “heteroalkyl” by itself or in combination with another term means, unless otherwise stated, a stable straight or branched chain radical consisting of the stated number of carbon atoms and one or two heteroatoms selected from the group consisting of O, N, and S, wherein the sulfur heteroatoms may be optionally oxidized and the nitrogen heteroatoms may be optionally quaternized or oxidized. The oxygens bonded to oxidized sulfur or nitrogen may be present in addition to the one or two heteroatoms in the heteroalkyl group. The heteroatom(s) may occupy any position in the heteroalkyl group, including the attachment position of the heteroalkyl group and a terminal atom of the heteroalkyl group. Examples of heteroalkyl groups include: ‘S—CH₂—CH₂—CH₃, —CH₂—CH₂CH₂—OH, —CH₂—CH₂—NH—CH₃, —CH₂—SO₂—NH—CH₃, —CH₂—S—CH₂—CH₃ and —CH₂CH₂—S(═O)—CH₃. Two heteroatoms may be bonded to each other, such as, for example, —CH₂—NH—OCH₃, or —CH₂—CH₂—S—S—CH₃.

The term “heterocycle” or “heterocyclyl” or “heterocyclic” by itself or as part of another substituent means, unless otherwise stated, an unsubstituted or substituted, stable, mono- or multicyclic heterocyclic ring system which consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

The term “heteroaryl” or “heteroaromatic” refers to a heterocycle having aromatic character. A monocyclic heteroaryl group is preferably a 5-, 6-, or 7-membered ring, examples of which are pyrrolyl, furyl, thienyl, pyridyl, pyrimidinyl and pyrazinyl. A polycyclic heteroaryl may comprise multiple aromatic rings or may include one or more rings which are partially saturated.

Examples of polycyclic heteroaryl groups containing a partially saturated ring include tetrahydroquinolyl and 2,3-dihydrobenzofuryl. For compounds according to Formula I, below, the attachment point on the aromatic group R² is understood to be on an atom which is part of an aromatic monocyclic ring or a ring component of a polycyclic aromatic which is itself an aromatic ring. For example, on the partially saturated heteroaryl ring, 1,2,3,4-tetrahydroisoquinoline, attachment points are ring atoms at the 5-, 6-, 7- and 8-positions. The attachment point on aromatic group R² may be a ring carbon or a ring nitrogen and includes attachment to form aromatic quaternary ammonium salts such as pyridinium.

Examples of non-aromatic heterocycles include monocyclic groups such as: aziridinyl, oxiranyl, thiiranyl, azetidinyl, oxetanyl, thietanyl, pyrrolidinyl, pyrrolinyl, imidazolinyl, pyrazolidinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, 1,4-dihydropyridinyl, piperazinyl, morpholinyl, thiomorpholinyl, pyranyl, 2,3-dihydropyranyl, tetrahydropyranyl, 1,4-dioxanyl, 1,3-dioxanyl, homopiperazinyl, homopiperidinyl, 1,3-dioxepinyl, 4,7-dihydro-1,3-dioxepinyl and hexamethyleneoxide.

Examples of monocyclic heteroaryl groups include, for example, six-membered monocyclic aromatic rings such as, for example, pyridyl, pyrazinyl, pyrimidinyl and pyridazinyl; and five-membered monocyclic aromatic rings such as, for example, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include: indolyl, indolinyl, quinolyl, tetrahydroquinolyl, isoquinolyl, 1,2,3,4-tetrahydroisoquinolyl, cinnolinyl, quinoxalinyl, quinazolinyl, phthalazinyl, 1,8-naphthyridinyl, 1,4-benzodioxanyl, chromene-2-one-yl (coumarinyl), dihydrocoumarin, chromene-4-one-yl, benzofuryl, 1,5-naphthyridinyl, 2,3-dihydrobenzofuryl, 1,2-benzisoxazolyl, benzothienyl, benzoxazolyl, benzthiazolyl, purinyl, benzimidazolyl, benztriazolyl, thioxanthinyl, benzazepinyl, benzodiazepinyl, carbazolyl, carbolinyl, acridinyl, pyrrolizidinyl and quinolizidinyl.

The term “heteroarylene,” by itself or as part of another substituent means, unless otherwise stated, a divalent heteroaryl radical. Preferred are five- or six-membered monocyclic heteroarylene. More preferred are heteroarylene moieties comprising divalent heteroaryl rings selected from the group consisting of pyridine, piperazine, pyrimidine, pyrazine, furan, thiophene, pyrrole, thiazole, imidazole and oxazole.

The aforementioned listing of heterocyclyl and heteroaryl moieties is intended to be representative, not limiting.

The terms “halo” or “halogen” by themselves or as part of another substituent, e.g., haloalkyl, mean, unless otherwise stated, a fluorine, chlorine, bromine, or iodine atom. Fluorine, chlorine and bromine are preferred. Fluorine and chlorine are most preferred.

The term “haloalkyl” means, unless otherwise stated, an alkyl group as defined herein containing at least one halogen substituent and no substituent that is other than halogen. Multiple halogen substituents, up to substitution of all substitutable hydrogens on the alkyl group may be the same or different. Preferred haloalkyl groups include, for example, perfluoro(C₁-C₆)alkyl, trifluoro(C₁-C₆)alkyl, gem-difluoro(C₁-C₄)alkyl and chloro(C₁-C₄)alkyl. More preferred haloalkyl groups include, for example, —CF₃, —C₂F₅, —CH₂CF₃, —CHF₂, CF₂CH₃ and —CH₂Cl.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “trifluoro(Cₓ-C_{y})alkyl” means an alkyl group with a minimum of x carbon atoms and a maximum of y carbon atoms, wherein the three hydrogen atoms on a terminal carbon (—CH₃) are replaced by fluorine atoms. Examples include —CH₂CF₃, —(CH₂)₂—CF₃ and —CH(CH₃)—CF₃.

The term “gem-difluoro(Cₓ-C_{y})alkyl” means an alkyl group with a minimum of x carbon atoms and a maximum of y carbon atoms, wherein one carbon atom is geminally substituted with two fluorine atoms. The fluorine-substituted carbon may be any carbon in the chain having at least two substitutable hydrogens, including the a terminal —CH₃ group and the proximal carbon through which the difluoro(Cₓ-C_{y})alkyl is bonded to the rest of the molecule. Examples include —CH₂CF₂H, —(CH₂)₂—CF₂H and —CF₂—CH₃ and 3,3-difluorocyclohexyl.

The term “substituted” means that an atom or group of atoms has replaced hydrogen as the substituent attached to another group. For aryl and heteroaryl groups, the term “substituted” refers to any level of substitution, namely mono-, di , tri-, tetra-, or penta-substitution, where such substitution is permitted. The substituents are independently selected, and substitution may be at any chemically accessible position.

The naming of compounds disclosed herein was done by employing the structure naming programs included in ChemDrawg software packages. The compounds, were named using the “Structure to Name” program within ChemDraw® Ultra Version 8.0 (© 1985-2003, CambridgeSoft Corporation, 100 Cambridgepark Drive, Cambridge, Mass. 02140 USA).

### DESCRIPTION

Methods of Treating or Preventing Viral Infection

The invention provides methods of treating viral infections, comprising administering at least one compound which is an inhibitor of cathepsin L to an individual in need of such treatment. The invention also provides methods of preventing viral infections, comprising administering at least one compound which is an inhibitor of cathepsin L to an individual likely to develop a viral infection. For either treatment or prevention, the viral infection is preferably SARS or Ebola and, most preferably, is SARS.

Inhibitors of Cathepsin L

Compounds useful in the methods of the invention include: compounds containing a valine-phenylalanine moiety; disulfide compounds according to Formula I; cyclopropenone compounds according to Formula II; 1-oxytriazole and 1-oxyimidazole compounds according to Formula III; ketone compounds according to Formula IV; aminoketone compounds according to Formula V; α-aminoketone compounds according to Formula VI; α-amino fluoroketone compounds according to Formulae VIIa, VIIb, VIIc and VIId, and pharmaceutically acceptable salts thereof, lactol compounds according to Formula VIIIa and VIIIb, and pharmaceutically acceptable salts thereof; L-tryptophanyl compounds according to Formula IX, and pharmaceutically acceptable salts thereof; sulfonamide compounds according to Formula X, and pharmaceutically acceptable salts thereof; epoxysuccinic acid compounds according to Formula XI, and pharmaceutically acceptable salts thereof, compounds according to Formula XII, having a heterocyclic leaving group, and pharmaceutically acceptable salts thereof; a polypeptide compound according having an amino acid sequence represented by Seq. ID No. 1; anti-cathepsin L antibodies; cysteine and serine protease inhibitors containing a ketomethylene group according to Formula XIII, having a ketomethylene group, and pharmaceutically acceptable salts thereof; compounds according to Formula XIV, having an indole nitrile group, and pharmaceutically acceptable salts thereof; N—[N-(L-3-trans-carboxyoxirane-2-carbonyl)-L-leucyl]-agmatine, Leupeptin, Z-Phe-Gly-NHO-Bz, Z-Phe-GIy-NHO-Bz-pOMe, Z-Phe-Gly-NHO-Bz-pMe, Z-Phe-Phe-FMK, Z-Phe-Tyr-CHO, Z-Phe-Tyr(t-Bu)-diazomethylketone, 1-Napthalenesulfonyl-Ile-Trp-CHO, Z-Phe-Tyr(OtBu)-COCHO, Z-Ill-FMK, Pindobind, L-3-carboxy-trans-2,3-epoxypropyl-leucyl-amide-(3-guanizino)butane (Ep-475), 1-(6-((8R,9S,1 3S,14S)-7,8,9,11,12,13,14,15,16,17-decahydro-3-methoxy-13-methyl-6H-cyclopenta[a]phenanthren-17-ylamino)hexyl)-1H-pyrrole-2,5-dione and 3,5 dinitrocatechol.

Isomerism in Compounds of the Invention

Compounds useful in the practice of the invention may comprise chiral centers which result in optical isomerism. The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. See March, Advanced Organic Chemistry, 4^{th} Ed., (1992), p. 109. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 3, the Cahn-Ingold-Prelog ranking is A>B>C>D. The lowest ranking atom, D is oriented away from the viewer.

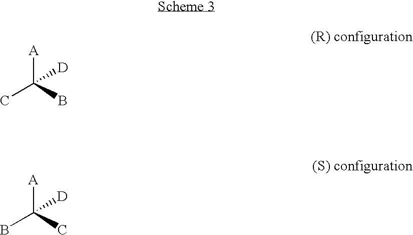

The method of the present invention is meant to encompass the use of compounds comprising optical isomers, as well as their racemic and resolved, diastereomerically and enantiomerically pure forms and salts thereof. Diastereomers result from the presence of more than one chiral center in a compound. Diastereomeric pairs may be resolved by known separation techniques including normal and reverse phase chromatography, and crystallization.

By “isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound having the structure of Formula I, or a chiral intermediate thereof, is separated into 99% wt. % pure optical isomers by HPLC using a suitable chiral column, such as a member of the series of DAICEL CHIRALPAK® family of columns (Daicel Chemical Industries, Ltd., Tokyo, Japan). The column is operated according to the manufacturer's instructions.

Preparation of Compounds According to the Invention

Compounds useful in the practice of the invention may be prepared via synthetic organic chemistry methods well known to one of ordinary skill in the are. See March, 1992, Advanced Organic Chemistry, John Wiley & Sons. Inc., New York, N.Y., 4th ed.; Stewart et al., 1984, Solid Phase Peptide Synthesis, Pierce Chemical Company, Rockford, Ill., 2^{nd} ed.; and Harlow et al., 1988, Antibodies, A Laboratory Manual, Cold Spring Harbor Laboratory Press, Cold Spring Harbor, N.Y.

In particular, compounds according to Formula I are prepared, for example, as follows.

Compounds of Formula I, wherein R¹ and R² are —N((C₁-C₆)alkyl)₂ may be prepared according to the method depicted in Scheme 4.

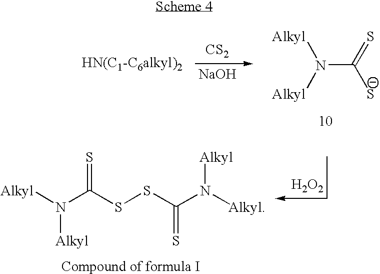

According to Scheme 4, the nucleophilic compound 10 is prepared by reaction of a dialkyl amine with carbon disulfide in the presence of sodium hydroxide. Compound 10 is then reacted with an oxidizing agent, preferably hydrogen peroxide to form the compound according to Formula I.

Compounds of Formula I, wherein R¹ and R² are C₃-C₁₀ hydrocarbyl, may be prepared according to the method depicted in Scheme 5.

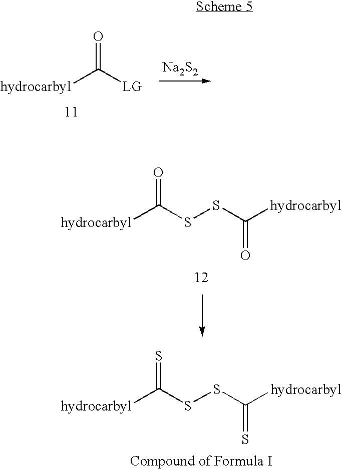

According to Scheme 5, intermediate compound 11, wherein LG is a leaving group, is reacted with sodium sulfide to form 12. Suitable leaving groups include halogen, preferably —Cl, and aryl, alkyl and haloalkyl sulfonated, preferably, methane sulfonyl, trifluoromethansulfonyl, 4-toluenesulfonyl, 4-nitrobenzenesulfonyl and benzenesulfonyl. The reaction is preferably performed in the presence of a suitable solvent. Preferred solvents include toluene, chloroform, methylene chloride, ethyl acetate, acetonitrile, ether and tetrahydrofuran. Compound 12 is reacted with Lawesson's reagent [19172-47-5] to form a compound of Formula I. Suitable protocols for the reaction with Lawesson's reagent are known. See, for example, Beil., 16, IV, 1113; Fieser, 1988, 13, 38; Fieser, 1990, 15, 37; and Fieser, 1992, 16, 37, the entire disclosures of which are incorporated herein by reference in their entirety.

Salts of Compounds According to the Invention

Compounds useful in the method of the present invention may take the form of salts. The term “salts,” embraces addition salts of free acids or free bases which are compounds of the invention. The term “pharmaceutically-acceptable salt” refers to salts which possess toxicity profiles within a range that affords utility in pharmaceutical applications.

Suitable pharmaceutically-acceptable acid addition salts may be prepared from an inorganic acid or from an organic acid. Examples of inorganic acids include hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, salicyclic, salicyclic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, toluenesulfonic, sulfanilic, cyclohexylaminosulfonic, stearic, algenic, γ-hydroxybutyric, salicyclic, galactaric and galacturonic acid.

Suitable pharmaceutically-acceptable base addition salts of compounds of the invention include for example, metallic salts including alkali metal, alkaline earth metal and transition metal salts such as, for example, calcium, magnesium, potassium, sodium and zinc salts. Pharmaceutically-acceptable base addition salts also include organic salts made from basic amines such as, for example, N,N′-dibenzylethylenediamine, chloroprocaine, choline, diethanolamine, ethylenediamine, meglumine (N-methylglucamine) and procaine. All of these salts may be prepared by conventional means from the corresponding compound according to Formula I by reacting, for example, the appropriate acid or base with the compound according to Formula I.

Dosage and Administration of an Inhibitor of Catheysin L

The invention also encompasses the use pharmaceutical compositions of a cathespsin L inhibitor to practice the methods of the invention, the compositions comprising an inhibitor of cathepsin L and a pharmaceutically-acceptable carrier.

As used herein, the term “pharmaceutically-acceptable carrier” means a chemical composition with which an inhibitor of cathepsin L may be combined and which, following the combination, can be used to administer an inhibitor of cathepsin L to a mammal.

The pharmaceutical compositions useful for practicing the invention may be administered to deliver a dose of between 1 ng/kg/day and 100 mg/kg/day. In one embodiment, the invention envisions administering a daily oral dose of 250 milligram to 1000 milligram of an inhibitor of cathepsin L to an individual with a viral infection. In one aspect of the invention, the inhibitor is tetraethylthioperoxydicarbonic diamide and the viral infection is one of SARS and Ebola. In another embodiment, the invention envisions administering an inhalation dose of 1 milligram to 250 milligram daily of an inhibitor of cathepsin L to an individual infected with a viral infection. In one aspect of the invention, the inhibitor is tetraethylthioperoxydicarbonic diamide and the viral infection is one of SARS and Ebola.

Pharmaceutical compositions that are useful in the methods of the invention may be administered systemically in oral solid formulations, ophthalmic, suppository, aerosol, topical or other similar formulations. In addition to an inhibitor of cathepsin L, such pharmaceutical compositions may contain pharmaceutically-acceptable carriers and other ingredients known to enhance and facilitate drug administration. Other possible formnulations, such as nanoparticles, liposomes, resealed erythrocytes, and immunologically based systems may also be used to administer an inhibitor of cathepsin L according to the methods of the invention.

Compounds which are identified using any of the methods described herein may be formulated and administered to a mammal for treatment of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

Methods of Identifying Potential Therapeutics for Viral Infection

In another embodiment, the invention provides methods to identify potential therapeutic compounds for the treatment of SARS or Ebola viral infection. The methods use either a lentiviral pseudotype system or a high throughput screening system of cathepsin L activity to identify modulators of cathepsin L activity. As demonstrated herein in the Examples, cathepsin L plays an important role in SARS CoV membrane fusion and viral entry. Inhibiting cathepsin L protealytic activity significantly reduces SARS or EboV entry into target cells. Modulators which reduce cathepsin L activity are therefore potential therapeutic compounds for use in the treatment or prevention of SARS infection or Ebola virus infection. The modulators are also extremely useful as research tools in further studying the role of cathepsin L activity in the entry of SARS or Ebola virus into target cells. Therefore, methods of identifying such modulators are useful.

The terms “modulating activity” “inhibiting activity” and “activating activity” of cathepsin L refer to the ability of a compound to activate or inhibit the proteolytic action of cathepsin L.

Modulation can be assayed by determining any parameter that is indirectly or directly affected by cathepsin L activity. In one preferred embodiment, modulation is measured using a lentiviral pseudotype system. Details of a preferred embodiment of a lentiviral pseudotype system are described in the Examples. In another preferred embodiment, modulation is measured using a high throughput screening assay. Details of a preferred embodiment of a high throughput screening assay are described in the Examples.

Test compounds can be any kind of molecule, including, but not limited to: peptides, peptidomimetics, nucleic acids, small molecules, or other drugs. The methods of the invention are not limited by the type of test compound used in the assay. The test compound may thus be a synthetic or naturally-occurring molecule, which may comprise a peptide or peptide-like molecule, or it may be any other molecule, either small or large, which is suitable for testing in the assay.

The test compounds of the present invention can be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145).

Examples of methods for the synthesis of molecular libraries can be found in the art, for example, in: DeWitt et al., 1993, PNAS 90:6909-6913; Erb et al., 1994, PNAS 91:11422-11426; Zuckermann et al., 1994, J. Med. Chem. 37:2678-2685; Cho et al., 1993, Science 261:1303-1305; Carell et al., 1994, Angew. Chem. Int. Ed. Engl. 33:2059-2061; Carell et al., 1994, Angew. Chem. Int. Ed. Engl. 33:2061-2064; and Gallop et al., 1994, J. Med. Chem. 37:1233-1251.

Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, PNAS 89:1865-1869), or phage (Scott and Smith, 1990, Science 249:386-390; Devlin, 1990, Science 249:404-406; Cwirla et al., 1990, PNAS 87:6378-6382; and Felici, 1991, J Mol. Biol. 222:301-310).

Compounds identified in vitro are then tested for activity against cathepsin L in vivo in animals. In one aspect, compounds are tested in non-human animals, preferably non-human mammals. In another aspect, compounds are tested in humans. Essentially, the compound is administered to the non-human animal or human by any of the routes described herein, and the effect of the compound is assessed by clinical and symptomatic evaluation. Such assessment is well known to the practioner in the field of infectious disease or those studying the pathogenic viruses.

Lentiviral Pseudotype System

The invention provides methods of identifying potential therapeutics for viral infection which inhibit cathepsin L. In one embodiment, the method uses a lentiviral pseudotype system that produces retroviral pseudotype incorporating the glycoprotein of either SARS-CoV or EboV. This system is described in detail using the SARS CoV S protein in Simmons et al., 2004, PNAS 101(12):4240-5, which is herein incorporated by reference in its entirety. In brief, HIV gag/pol is co-expressed in 293T cells together with SARS S of EboV GP glycoprotein. The resulting HIV virions released into the culture supernatant incorporate S or GP into their lipid coat. These pseudovirions infect target cells in a S or GP dependent manner. Cell tropism mimics live SARS-CoV or EboV, and infection can be inhibited by patient sera and specific monoclonal antibodies raised against S or GP. The lentiviral pseudotype system can use reporter gene expression to measure viral entry into a target cell. Reporter gene expression is proportional to viral entry. Thus, a test compound that inhibits viral entry results in reduced reporter gene expression compared to reporter gene expression in the absence of the test compound. Consequently, the system can be used for discovery of inhibitors by coincubation of compounds from a compound library, or any other source, with the target cells and pseudotype virions.

The viral pseudotypes described herein offer a unique tool for use in high throughput screening (HTS) for inhibitors of SARS CoV S or EboV GP mediated infection. Unlike SARS CoV or EboV, these pseudotypes advantageously do not require high biosafety containment and are thus amenable to use with robotic screening. Additionally, reporter genes useful for high throughput screens can be encoded by the pseudotyped virus, further facilitating analysis of large compound libraries.

Sequences for various strains of SARS CoV S glycoprotein and EboV GP glycoprotein are known to the artisan. Non-limiting examples include: SEQ ID NO. 2 (the coding sequence for the Urbani strain SARS S protein) and SEQ ID NO.3 (the coding sequence for the Zaire strain EboV GP protein).

Target cells for use in the method include any cell that is susceptible to viral entry by the virus of interest. For SARS CoV, suitable target cells are any cell that expresses receptor ACE2. The target cells can express ACE2 endogenously or can be recombinant cells expressing ACE2 via heterologous expression. Since the protease activity of ACE2 is not required for SARS CoV S entry (Li et al., 2003, Nature, 426(6965):450-4 ), recombinant cells expressing mutants of ACE2 are also suitable target cells. Suitable target cells include, but are not limited to, 293T (human; Hu), A54 (Hu), HeLa (Hu), HT1080 (Hu), Huh-7(Hu), NP2 (Hu), COS (African green monkey; AGM), and Vero (AGM). Particularly preferred cells are 293T, HT1080, Huh-7 and Vero and more preferred are 293T and Vero cells. Target cells for EboV include, but are not limited to, airway epithelial cells, vascular endothelial cells, Vero (AGM), Vero E6, 293T (Hu), HepG2 (Hu), primary human liver cells, HeLa (Hu), BSC-1 (simian; Si), Cos-7 (Si), NIH 3T3 (murine), MDBK (bovine, Bo), BAEC (Bo), CHO (Hamster), QT6 (Quail), U87 (Hu), MDCK (canine). Particularly preferred for EboV are 293T, Vero (AGM), Vero E6, HepG2 (Hu), and primary human liver cells.

To screen for inhibitors of infection by SARS CoV, cells expressing ACE2 are challenged with a SARS S pseudotype virus in the presence of a test compound. In one embodiment, the pseudotype virus is HIV-luc(SAR S). To screen for inhibitors of infection by EboV, target cells of EboV are challenged with a Ebola GP glycoprotein pseudotype virus in the presence of a test compound. About twenty four to about forty hours after infection, cellular lysates are prepared and assayed for reporter gene activity. A test compound which reduces the amount of expression of the luciferase compared to the system in the absence of the test compound is identified as a potential therapeutic for treatment of SARS CoV or EboV infections.

The skilled artisan knows the types of control infections to perform to confirm the analysis. For instance, control infections with VSV-G pseudotypes should not be affected by compounds specific for SARS CoV S, but will illuminate those which are generally toxic to cells, generally affect the endosomal compartment, inhibit retroviral proteins, or affect expression of the luciferase reporter gene. Additional controls can include positive controls, such as the cathepsin L inhibitors E-64c and Z-LLL-FMK, identified herein as potent inhibitors of SARS CoV S-mediated infection. Negative controls can also be used, such as compounds that affect endosomal proteases other than cathepsin L (e.g. CA-074 or aprotinin).

Additionally, the specificity of any potential inhibitors for SARS CoV S or EboV can be confirmed by analyzing the affect of any compounds found in the primary screen using a different retroviral pseudotype system with a different reporter gene. This ensures that the effects seen are directed at the glycoprotein or the virus itself. For SARS CoV, confirming the effects on the S glycoprotein can be done using murine leukemia virus S pseudotypes encoding R-galactosidase or, for effects on SARS CoV itself, use of BSL-3 conditions and scoring for plaque reduction. Although the luciferase reporter system, a preferred embodiment, is highly sensitive and linear over a wide range, it requires lysis of the cells. In another embodiment, the high throughput screen uses secreted alkaline phosphatase (SEAP).

High Throughout Screening Using Microarrays

A methodology to screen chemical libraries on microarrays has been developed (Gosalia et al., 2003, PNAS 100(15):8721-6 incorporated herein by reference in its entirety). In brief, chemical compounds within individual nanoliter droplets of glycerol are microarrayed onto glass slides at 400 spots/cm². Using aerosol deposition, subsequent reagents and water are metered into each reaction center in order to rapidly assemble diverse multicomponent reactions without cross-contamination or the need for surface linkage. This proteomics technique allows for the kinetic profiling of protease mixtures, protease—substrate interactions, and high throughput screening reactions.

A preferred method of this technology is described in the Examples.

In a preferred embodiment, the method uses cathepsin L and a cathepsin L substrate and assays test compounds for inhibition. The cathepsin L is preferably human. The substrate is a peptide or protein substrate, fluorogenic substrate, or calorimetric substrate. Preferably, the substrate is fluorogenic or colorimetric, allowing for visual detection of the extent of the cathepsin L-catalyzed cleavage reaction. An exemplary substrate for cathepsin L is VEID-MCA.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Experimental Example 1

### Receptor-Induced Proteolvtic Activation of SARS Coronavirus Spike Glycoprotein Membrane Fusion

The materials and methods used in the experiments presented in this Experimental Example are now described.

Cell lines, plasmids and antibodies: Human ACE2 was amplified from a human cDNA library (Invitrogen), using primers designed to the beginning and end of ACE2 and cloned into pcDNA3.1. ACE2 sequence contained one coding difference (Q to L change at position 24) to that previously published (Tipnis et al., 2000, J. Biol. Chem. 275:33238-33243). pCAGGS SARS-CoV S, and plasmids expressing VSVG, MLV-Amphotropic envelope and ASLV-A envelope have been previously described (Gilbert et al., 1994, J Virol 68:5623-5628; Simmons et al., 2004, PNAS 101:4240-4245).

All cell lines were maintained in DMEM10 (DMEM supplemented with 10% fetal bovine serum). Stable HeLa/Tva cells were produced using pcDNA6 encoding Tva and carrying a blasticidin resistance marker. Following selection using blasticidin, clones were single cell cloned by limiting dilution and assayed for efficient Tva expression by flow cytometry. 293T cells were transiently transfected with human ACE2, control plasmid or SARS-CoV S protein using standard calcium phosphate transfection techniques and challenged 48 hours post-transfection.

For the detection of S protein fragments, two antisera were employed. IMG-557 (Imgenex, San Diego, Calif.) is a polyclonal rabbit sera raised to a peptide corresponding to amino-acids 1124-1140 of S protein, while IMG-5010 (Imgenex, San Diego, Calif.) is a murine monoclonal antibody raised to a peptide corresponding to amino-acids 19-35.

Pseudotype preparation: 293T cells were seeded into 10 cm dishes to be approximately 70% confluent the following day. Cells were transfected by standard calcium phosphate transfection techniques with 10 μg of HIV gag/pol encoding either luciferase or GFP (pNL-luc or PNL-GFP) (Connor et al., 1995, Virology 206: 935-944) and 30 μg of viral envelope, ACE2 or control plasmid. Dual envelope expressing virions were transfected with 10 μg pNL-GFP, 15 μg ASLV-A envelope and 20 μg SARS-CoV S protein. Cells were washed the next day and then induced with sodium butyrate (10 mM) for 5 hours before further washing. 16 hours later cell supernatant was filtered through a 0.45 μm pore size screen.

If required, virions were concentrated by ultracentrifuge concentration at 40,000 rpm in a SW41 rotor through a 20% sucrose cushion for 1 hour at 4° C. Pellets were then allowed to resuspend in PBS with calcium and magnesium overnight at 4° C.

Trypsin pretreatment: Concentrated pseudovirions from one plate of transfected 293T cells were treated with increasing concentrations of TPCK-trypsin (Sigma) for 10 minutes at 25° C. Trypsin was then inhibited by addition of DMEM10 supplemented with 75 μg/ml soybean trypsin inhibitor (Sigma). Samples were then either lysed in RIPA buffer for SDS-PAGE analysis or used to spin infect 293T cells transiently transfected with human ACE2 at 1200×g for 2 hours at 4° C. Following incubation for 5 hours at 37° C., medium was changed and cells were incubated for a further 40 hours. Cells were then analyzed for luciferase activity using a commercial assay as per manufacturer's instructions (Promega).

Blots were probed with a murine monoclonal antibody (IMG-5010) raised against a peptide corresponding to residues 19-35 of SARS-CoV S protein (Imgenex, San Diego, Calif.), or polyclonal rabbit serum (IMG-557) raised against a peptide corresponding to residues 1124-1140 of SARS-CoV S protein (Imgenex, San Diego, Calif.).

Trypsin bypass: 293T cells transiently expressing ACE2 were pre-incubated at 37° C. for 45 minutes with DMEM10 or DMEM10 containing ammonium chloride (20 mM). Medium was then replaced with cold DMEM10 or cold DMEM10 containing ammonium chloride (40 mM) and incubated for a further 15 minutes at 4° C. An equal volume of diluted cold pseudotype supernatant was added (1 in 10 dilution of wild-type S and S(R667A) and 1 in 100 dilution of VSV-G) and cells were spin infected at 4° C. as described above to allow virus binding to cells but not endocytosis or membrane fusion. Following spin infection, medium was replaced with warm PBS or warm PBS containing ammonium chloride (20 mM) and incubated at 37° C. for 15 minutes to allow any conformational rearrangements that may be necessary in S protein. PBS was then removed and fresh PBS or PBS containing TPCK-trypsin (15 μg/ml) was added for 10 minutes at 25° C. PBS was removed and DMEMIO supplemented with soybean trypsin inhibitor (75 μg/ml) with or without ammonium chloride (20 mM) was added. Medium was replaced with DMEM10 lacking ammonium chloride 12 hours later. Cells were then analyzed for luciferase activity after a further 36 hours.

Protease inhibitors: 293T cells or Vero E6 cells were pre-treated for 1 hour with increasing concentrations of leupeptin (Roche Biochemicals), CA-074 (Sigma) or Z-leu-leu-leu-fluoromethyl ketone (Z-LLL-FMK) (Sigma). Inhibitors were removed and replaced with the same inhibitors at double the final concentration. An equal volume of pseudotypes were then added and cells were spin infected as described above. Following spin infection the cells were incubated for 5 hours, then medium was replaced with fresh DMEM10 without drug. Cells were assayed for luciferase activity after 40 hours.

Replication-competent SARS-CoV assays: SARS-CoV(strain Tor2) was handled under BSL3 conditions. Virus passaged 6 times in Vero E6 cells and titered on Vero E6 cells by limiting dilution and scoring for presence of cytopathic effect. For trypsin bypass experiments Vero E6 cells were incubated on ice for 1 hour with medium alone or medium containing 25 mM ammonium chloride or 500 μg/ml leupeptin. SARS-CoV at a multiplicity of infection (MOI) of approximately 0.5 was then added and cells were spin infected at 4° C. for 1 hour at 1200×g. Virus was removed and cells were incubated for 10 minutes with serum-free DMEM at 37° C. The medium was then replaced with fresh medium or DMEM containing TPCK-trypsin at 15 microgram/ml and cells were incubated at room temperature for 10 minutes. Trypsin was removed and replaced with medium containing soybean trypsin inhibitor (75 μg/ml) and the relevant concentration of ammonium chloride or leupeptin. Cells were incubated at 37° C. for 4 hours, at which time medium was replaced with fresh medium without inhibitors and cells were incubated for a further 40 hours. Cells were fixed for 10 minutes in cold methanol: acetone and incubated for 2 hours at 65° C. Cells were then immunostained with anti-S protein antibodies IMG-557 and IMG-5010 at 0.5 μg/ml followed by a mixture of anti-rabbit and anti-mouse FITC conjugates. For leupeptin sensitivity assays 293T cells transiently overexpressing ACE2 were pre-treated for 1 hour with increasing concentrations of leupeptin or medium alone. Leupeptin was removed and replaced with fresh leupeptin at double the final concentration and the cells were challenged with an equal volume of virus at a MOI of approximately 5. Following 3 hours of incubation, cells were washed twice and incubated with either fresh medium or increasing concentrations of leupeptin for a further 4 hours. Medium was then replaced with fresh medium without inhibitor and cells were incubated for 72 hours. Supernatant was harvested and spun to remove cell debris and then lysed in a final concentration of 1% Empigen (Calbiochem). Following heat inactivation at 65° C. for 1 hour, samples were analyzed for SARS-CoV nucleocapsid using a commercial ELISA kit (Imgenex, San Diego, Calif.).

Characterization of HIV-luc(ACE2) particles: 293T cells transiently transfected with control plasmid, ACE2 or SARS-CoV S protein were challenged using spin infection as above with pseudotype particles normalized using an in-house p24 assay to 3000 ng p24/ml. Cells were assayed for luciferase activity after 48 hours.

Intervirion fusion: Concentrated HIV-luc(ACE2) virus particles were mixed with concentrated HIV-GFP particles incorporating ASLV-A envelope, SARS-CoV S protein or both envelopes in PBS. Approximately 90 ng p24 of HIV-luc(ACE2) and 180 ng p24 of HIV-GFP viruses were used per assay in a total of 100 μl. The viruses were allowed to bind at 4° C. for 30 minutes, followed by 15 minutes at 37° C. for conformational rearrangements to occur. Virions were then adjusted to the desired pH using 0.1 M citric acid. 25 μl of PBS, TPCK-trypsin (final concentration 10 μg/ml), pre-activated cathepsin B or L (final concentrations 2 pg/ml) or cathepsin L buffer alone was then added. Recombinant cathepsin L (R&D Systems) was activated by incubation for 15 minutes at 10 μg/ml in 50 mM 2-(N-morpholino)ethanesulfonic acid (MES), pH 6.0 on ice. Recombinant cathepsin B (R&D Systems) was pre-activated in 25 mM MES, 5 mM DTT, pH 5.0 for 30 minutes at 25° C. After a 10 minute incubation at 25° C., proteolysis was halted by addition of 300 μl of DMEM10 containing leupeptin (25 μg/ml). Virions were then incubated at 37° C. for 30 minutes to allow membrane fusion to occur. 100 p1 of the virion mixture was then added in quadruplicate to HeLa/Tva cells pre-treated for 1 hour with leupeptin (20 μg/ml). Cells were spin infected as described above and incubated at 37° C. for 5 hours. Medium was then replaced with fresh DMEM10 without leupeptin and cells were assayed for luciferase activity 40 hours later.

Temperature sensitivity intervirion fusion assay: Intervirion fusion assays were performed as described above, except binding was performed wholly at 4° C. for 45 minutes for some samples, while others were allowed to bind at 4° C. for 30 minutes, followed by 15 minutes at 37° C. for conformational rearrangements to occur. Those incubated at 37° C. were allowed to equilibrate back to 4° C. to 5 minutes, then cold TPCK-trypsin was added to a final concentration of 10 μg/ml. Following a 15 minute incubation at 4° C., proteolysis was halted by the addition of DMEM10 supplemented with soybean trypsin inhibitor (75 μg/ml) and leupeptin (25 μg/ml). Virions were then incubated at 37° C. for 30 minutes to allow membrane fusion to occur and the assay was completed as described above.

The results of the experiments presented in this Experimental Example are now described.

Proteolysis Activates SARS-CoV S Protein Membrane Fusion Potential and Overcomes a Lysosomotropic Block

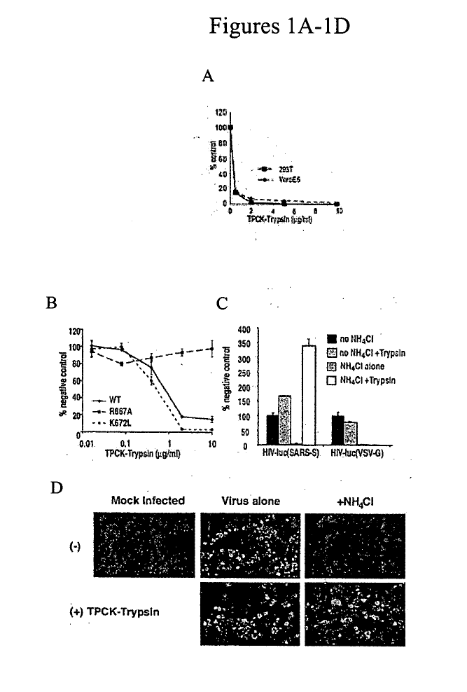

Fusion between Vero E6 cells and 293T cells expressing SARS-CoV S protein is greatly enhanced at neutral pH by trypsin activation of S protein, suggesting that SARS-CoV can enter cells by a pH-independent mechanism. However, lysosomotropic agents block SARS-CoV infection, suggesting that acid pH can also play a role in virus entry (Simmons et al., 2004, PNAS 101:4240-4245). To resolve this apparent discrepancy, it was first determined if trypsin treatment also enhances viral infectivity. To do this, retroviral pseudovirions bearing the SARS-CoV S protein were treated with different concentrations of trypsin (FIG. 1A). When retrovirus SARS-CoV S pseudotypes were incubated with even very low concentrations of TPCK-trypsin, viral infectivity was greatly reduced (FIG. 1A). However, if virions were first allowed to bind to the cell surface, subsequent incubation with TPCK-trypsin actually enhanced viral infectivity (FIG. 1C). Similarly, cell-to-cell fusion was greatly enhanced if cells were exposed to trypsin after binding of S protein expressing effector cells to target cells overexpressing ACE2, the receptor for SARS CoV, compared to trypsin treatment of either effectors or targets prior to mixing (data not shown). Thus, trypsin treatment can enhance SARS CoV S protein mediated fusion both in the context of cell-cell and virus-cell membrane fusion.

One of the hypotheses developed to reconcile the apparent requirement of acid pH for virus infection with the ability of trypsin-activated SARS Co-V to mediate cell-cell fusion at neutral pH posited that exogenous trypsin cleavage mimics the action of a pH-dependent protease that cleaves the SARS CoV S protein after it is internalized and delivered to endosomes: If true, this hypothesis predicts that trypsin treatment of cell-associated virus could overcome the block to viral entry mediated by the lysosomotropic agent ammonium chloride. As previously demonstrated, pretreatment of cells with ammonium chloride dramatically reduced infection mediated by S protein (FIG. 1C) as well as by the pH-dependent viral glycoprotein, VSV-G. However, if SARS-CoV S protein pseudotypes bound to the cell surface were trypsin activated, infection occurred in the presence or absence of ammonium chloride (FIG. 1C). In fact, the combination of trypsin activation coupled with ammonium chloride increased viral infectivity by 3-fold. Thus, trypsin treatment of the SARS S glycoprotein not only enhances its ability to mediate membrane fusion, but also relieves it of a requirement for acid pH during the viral entry process. Similarly, trypsin treatment of live, replication-competent SARS-CoV bound onto Vero E6 cells, overcame the block to viral infection otherwise mediated by ammonium chloride (FIG. 1D).

Sensitivity of SARS-CoV S Protein to Tryptic Digestion

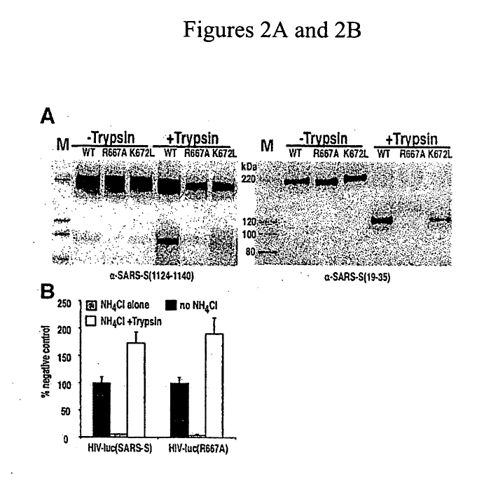

The ability of mild proteolysis to bypass the SARS-CoV infection requirement for low pH and to activate the membrane fusion potential of the S protein prompted us to examine the effect of trypsin treatment on S. On the 4-15% acrylamide Tris-HCl gels used in this study, uncleaved S protein migrated consistently at a position corresponding to a mass somewhat over 200 kDa (FIG. 2A). TPCK-trypsin digestion of HIV(SARS-S) pseudotyped virions generated a C-terminal S protein fragment of approximately 100 kDa that could be detected using either antibodies to the C-terminal V5 tag (Simmons et al., 2004, PNAS 101:4240-4245) or polyclonal sera raised against a peptide corresponding to the C-terminal extracellular portion of S protein (FIG. 2A left panel). A monoclonal antibody directed to the very N-terminus of mature S protein revealed an N-terminal tryptic fragment of approximately 120 kDa (FIG. 2A right panel). Thus, trypsin treatment appeared to cleave the S protein efficiently at a single position, generating both N— and C-terminal fragments. This is analogous to other coronavirus S glycoproteins which often undergo a posttranslational proteolytic cleavage that results in the production of S1 and S2 protein subunits.

Trypsin Inactivation, but Not Activation, Occurs at the Predicted S1/S2 Boundary

The sizes of the fragments liberated by trypsin proteolysis of S protein (FIG. 2A) are consistent with cleavage at one of two basic residues (arginine at position 667 and lysine at position 672) located at the predicted S1/S2 boundary based on alignments with other coronavirus S proteins (Bergeron et al., 2005, Biochem Biophys Res Commun 326:554-563). Indeed, mutation of the arginine at position 667 to an alanine (termed S(R667A)), but not the lysine at position 672 (S(K672L)), led to a loss of the major trypsin cleavage products (FIG. 2A), although a larger N-terminal fragment was observed, consistent with less efficient proteolysis C-terminal to arginine 667. Even in the absence of trypsin, a basal level of cleavage of S protein to yield the 100 kDa C-terminal fragment occurs (FIG. 2A), possibly during viral release from producer cells. Indeed, Bergeron and colleagues have also noted the inefficient cleavage of S protein in cell lysates resulting in a C-terminal fragment corresponding to approximately 110 kDa (Bergeron et al., 2005, Biochem Biophys Res Commun 326:554-563). Furthermore, introduction of a consensus furin cleavage site at amino-acid 667 leads to more efficient production of this fragment (Bergeron et al., 2005, Biochem Biophys Res Commun 326:554-563). Thus, it is likely that pre-treatment of S protein with trypsin results in cleavage at arginine 667 and the liberation of a C-terminal fragment of 100-110 kDa in size. Pseudotypes bearing S(R667A) remained fully infectious (FIG. 1B) and following attachment to cells could be induced to overcome the ammonium chloride mediated block to infection by cleavage with trypsin (FIG. 2B). However, unlike wild-type S protein and a second mutant (S(K672L)), pre-treatment of virions bearing S(R667A) with trypsin did not result in their inactivation (FIG. 1 B). Thus, while trypsin is able to cleave and inactivate SARS-CoV S protein at the predicted S1/S2 boundary located at position 667, this motif is not required for S activation associated with proteolysis of S protein posited to occur following endocytosis.

Sensitivity of SARS-CoV S Protein Mediated Entry to Protease Inhibitors

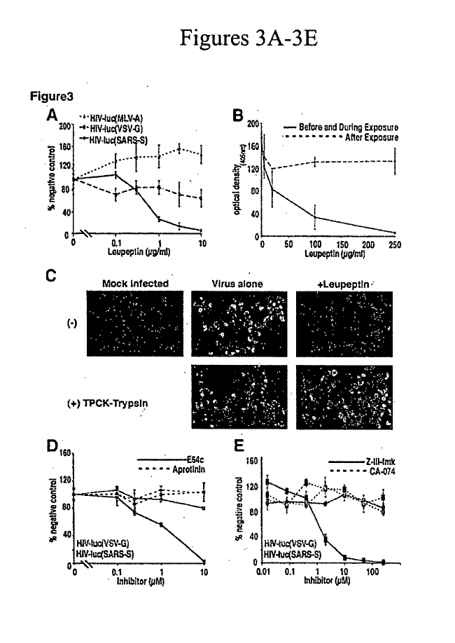

The ability of trypsin treatment to overcome ammonium chloride inhibition of SARS-CoV S glycoprotein-mediated infection suggests that lysosomotropic agents may prevent proteolytic processing of S glycoprotein by endosomal, pH-dependent proteases. To test this hypothesis, S protein-mediated pseudotype infection of 293T cells was examined in the presence of leupeptin, an inhibitor of endosomal trypsin-like serine and cysteine proteases (FIG. 3A). Entry mediated by SARS-CoV S protein was efficiently blocked by leupeptin, with over 95% inhibition observed at 10 μg/ml. Infection mediated by VSV-G, a pH-dependent viral membrane fusion protein, as well as the pH-independent envelope, MLV-Amphotropic Env, was not inhibited by leupeptin (FIG. 3A). Specific inhibition of S protein-mediated entry into Vero E6 cells was also observed, although higher concentrations of leupeptin were required, with 100 μg/ml necessary in order to achieve 95% inhibition (data not shown).

In addition to replication-deficient pseudovirions, infection of 293T cells transiently expressing ACE2 by replication-competent SARS-CoV was also inhibited by leupeptin (FIG. 3B). Efficient inhibition of infection was only observed if leupeptin was present 1 hour before and during the 3 hour exposure to virus. If leupeptin was added to cells following exposure to SARS-CoV and then removed 4 hours later, there was little or no effect on SARS-CoV replication, even at a concentration of 250 μg/ml. Thus, it is unlikely that the concentrations of leupeptin required to efficiently inhibit a spreading SARS-CoV infection are inhibiting post-entry steps of replication or are merely toxic to the cells. Rather, leupeptin appears to inhibit an early step in viral entry. In a similar manner to inhibition by ammonium chloride (FIG. 1), the leupeptin-mediated block to SARS-CoV infection of Vero E6 cells could be bypassed by treatment of virus bound to the cell surface with trypsin (FIG. 3C). Thus, it appears that trypsin cleavage is able to compensate for cleavage of S protein mediated by leupeptin sensitive endosomal proteases.

Leupeptin inhibits both serine and cysteine proteases. Therefore, more specific protease inhibitors were examined for their effects on S protein-mediated virus entry. E64c, an inhibitor of cysteine proteases, specifically inhibited infection by pseudotypes bearing S protein, while aprotinin, an inhibitor of serine-type proteases, had no effect (FIG. 3D). Inhibitors of other classes of proteases such as pepstatin, an aspartate protease inhibitor, also had no effect on either S protein or VSV-G mediated infection (data not shown). Finally, the specificity of endosomal cysteine proteases in mediating SARS-CoV infection was further examined in Vero E6 cells using inhibitors of specific cysteine proteases. CA-074, a selective inhibitor of cathepsin B (Montaser et al., 2002, Biol Chem 383:1305-1308) and Z-leu-leu-leu-fluoromethyl ketone (Z-LLL-FMK), an inhibitor of both cathepsin B and L (Hanisch et al., 2003, Eur J Immunol 33:3242-3254) showed differential inhibition of S protein-mediated entry (FIG. 3E). Z-LLL-FMK efficiently inhibited infection of HIV-luc(SARS S), but not HIV-luc(VSV-G), while CA-074 did not dramatically affect infection of either pseudovirus. Similar results were observed in both parental 293T cells and 293T cells overexpressing ACE2 (data not shown). These results suggest that cathepsin L, but not cathepsin B, plays a critical role in SARS-CoV S protein-mediated entry.

Protease-Mediated Activation of Membrane Fusion

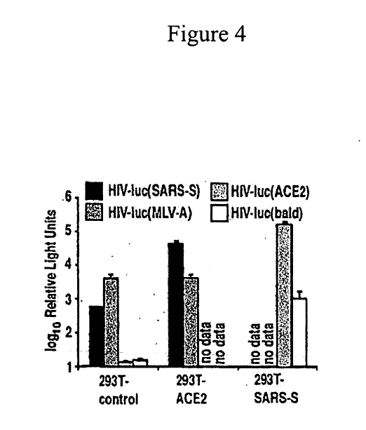

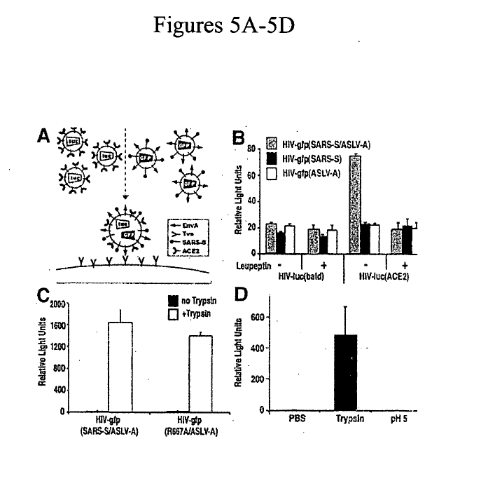

To further study the relative contributions of acid pH and proteolytic activation for S protein-mediated membrane fusion, a cell-free, virus-virus fusion assay was developed. To do this, HIV gag/pol and ACE2 were transiently expressed in 293T cells, which resulted in the efficient production of HIV particles containing ACE2 in their lipid coats as determined by western analysis of purified virions (data not shown). These ACE2 pseudotyped HIV particles encoding luciferase, HIV-luc(ACE2), were able to efficiently and specifically infect 293T cells transiently expressing the SARS-CoV S protein (FIG. 4). Fusion was absolutely dependent upon the presence of the SARS-CoV S protein. Particles produced without co-transfection of ACE2, or “bald” particles (termed HIV-luc(bald)), were also able to specifically infect 293T-SARS S cells, but at a level two logs lower than HIV-luc(ACE2) particles. Infection by these particles was presumably due to incorporation of low-levels of endogenous ACE2 from the 293T producer cells into the particles since their infection was strictly dependent upon SARS S expression in the target cells (FIG. 4).

In addition to the receptor-bearing HIV-luc(ACE2) particles, HIV particles encoding GFP and incorporating both the SARS-CoV S protein and the envelope glycoprotein from avian sarcoma and leukosis virus subtype A (ASLV-A env) were also produced. These double pseudotyped particles are referred to as HIV-gfp(SARS S/ASLV-A). The receptor and Env-bearing viral pseudotypes were then added individually or together to HeLa cells that lacked ACE2 but which expressed the receptor for ASLV-A, Tva (FIG. 5A). The HIV-gfp(SARS S/ASLV-A) particles readily infected the HeLa-Tva cells, while the HIV-luc(ACE2) particles did not. However, when both particles were added to the HeLa-Tva cells, both GFP and luciferase were expressed at high levels (FIG. 5B). Since the HIV-gfp(SARS S/ASLV-A) express only GFP, S protein/ACE2 mediated virus-to-virus fusion must occur in order for ASLV-A Env mediated transfer of the luciferase reporter gene into the target cells to occur. Thus, luciferase activity is a measurement of SARS CoV S protein-mediated intervirion membrane fusion.

This virus-virus membrane fusion assay was used to examine the effects of proteolytic activation of S protein on the membrane fusion process. It was found that if the target HeLa/Tva cells were pretreated with leupeptin, then S protein-mediated fusion was lost and luciferase activity returned to background levels (FIG. 5B). Leupeptin was found to have no effect on ASLV-A Env mediated infection of HeLa/Tva cells (data not shown). These results suggest that, in contrast to the model (FIG. 5A), for virus-virus membrane fusion to occur, the particles must be co-endocytosed into endosomes where proteases sensitive to leupeptin, such as cathepsin L, are able to act upon S protein, making fusion between the virus particles possible. Thus, in subsequent assays target cells were pre-treated with leupeptin in order to determine the effect of addition of exogenous protease on virus-virus fusion prior to plating on target cells. To more directly assess the requirement for proteolytic activation of S protein, the two pseudovirion populations were incubated to allow S protein and ACE2 mediated virus-virus binding. TPCK-trypsin treatment of the bound virus particles dramatically increased luciferase expression in target HeLa/Tva cells where endosomal proteolysis was blocked with leupeptin (FIG. 5C). Furthermore, fusion mediated by double pseudotyped virus bearing the mutant S(R667A), in addition to ASLV-A Env, was also enhanced by trypsin proteolysis confirming that cleavage at the predicted S1/S2 boundary is not required for fusion activation. In contrast to TPCK-trypsin, a brief low-pH pulse did not facilitate virus-virus fusion as assayed by luciferase gene transfer in the leupeptin treated target cells (FIG. 5D). These results further confirm that a low pH environment does not act as a direct trigger for SARS-CoV entry. Rather, these data are most consistent with a model in which the low pH environment of the endosome is needed for proteolytic activation of the SARS CoV S protein membrane fusion activity.

Activation of S Protein Membrane Fusion by Cathepsin L

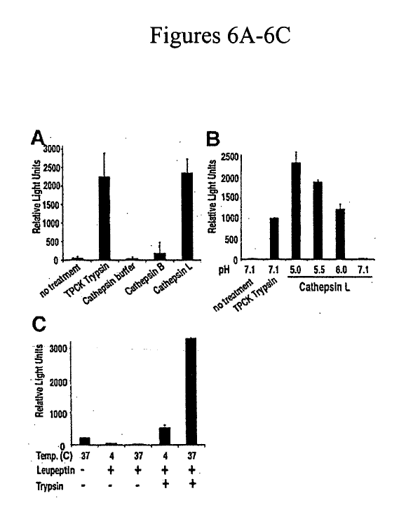

Our studies with protease inhibitors suggested that cathepsin L may play a role in promoting efficient S protein-mediated virus entry (FIG. 3). Therefore, recombinant cathepsins were used to activate virus particles in place of TPCK-trypsin. Treatment of mixed HIV-luc(ACE2) and HIV-gfp(SARS S/ASLV-A) particles with cathepsin L at pH 6.0 was able to mediate intervirion fusion as efficiently as TPCK-trypsin (FIG. 6A). In contrast, cathepsin B treatment did not produce a reproducible increase in intervirion fusion. These findings agree with the inhibitor results (FIG. 3), which suggested that cathepsin L, but not cathepsin B was involved in S protein activation. Given that cathepsin L is ubiquitously expressed, it is likely that cathepsin L plays a major role in S protein activation within endosomes. The sensitivity of SARS-CoV S protein-mediated entry to lysosomotropic agents is likely explained by the fact that cathepsin L and similar endosomal proteases cleave more efficiently, and are more stable, at acidic pH. To address this, cathepsin L-mediated activation of intervirion fusion was carried out at different pH's. A gradual reduction in levels of fusion was observed with increasing pH, and incubation at pH 7.1 resulted in no intervirion fusion (FIG. 6B). As was observed with TPCK-trypsin (FIGS. 1A and 1B), in order for cathepsin L to efficiently activate S protein, the particles had to be first incubated with ACE2, either in the context of ACE2-containing pseudovirions or ACE2-expressing cells.

Temperature Dependence of Protease Activation

The fact that S protein needs to bind ACE2 in order for either cathepsin L or TPCK-trypsin treatment to efficiently activate the protein suggested that conformational changes induced by receptor binding may be required prior to proteolytic activation. Conformational changes in envelope proteins are generally slowed or arrested at low temperatures. Thus, it was examined whether incubation of mixtures of HIV-luc(ACE2) and HIV-gfp(SARS S/ASLV-A) virions at 4° C. compared to 37° C. prior to treatment with protease affected subsequent membrane fusion activity, possibly by preventing conformational changes in S protein induced by ACE2 binding. Preliminary studies suggested that efficient cathepsin L cleavage of S protein did not occur below 15° C. (data not shown). Therefore, the temperature-dependence of intervirion fusion was studied using TPCK-trypsin, which cleaved S efficiently at 4° C. Only a small increase in intervirion fusion was seen with HIV-luc(ACE2) and HIV-gfp(SARS S/ASLV-A) virus particles maintained at 4° C., despite TPCK-trypsin treatment (FIG. 6C). If the mixture of HIV-luc(ACE2) and HIV-gfp(SARS S/ASLV-A) particles were pre-incubated at 37° C. for 15 minutes, however, before being returned to 4° C. for TPCK-trypsin treatment, efficient intervirion fusion was observed (FIG. 6C). These results indicate that a receptor and temperature-dependent step occurs prior to protease cleavage of S protein, possibly involving receptor-induced conformational changes within S protein to either expose a protease cleavage site or to undergo some of the steps leading up to membrane fusion.

A discussion of the results presented above is now presented.

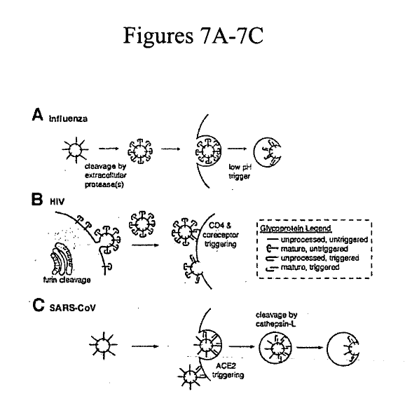

Many viral membrane fusion proteins can be categorized as either class I or class II based on their structure and mode of action. The majority of class I fusion proteins, as exemplified by influenza hemagglutinin (HA), associate as trimers prior to being cleaved by host proteases to form trimeric heterodimers. This cleavage can occur either at a late step of biosynthesis by furin-like proteases as is the case for HIV envelope (Env), or at the cell surface or in the extracellular matrix as is seen for HA (see FIG. 7). Cleavage induces a metastable state within the envelope glycoprotein, allowing triggers, such as receptor binding for pH-independent viruses or the acidic environment of the endosome for pH-dependent viruses, to activate conformational rearrangements leading to the highly stable six-helix bundle formation within the transmembrane subunit (Carr et al., 1997, PNAS 94:14306-14313; Chen et al., 1998, Cell 95:409-417). In the majority of class I viral fusion proteins cleavage is additionally required in order to expose the fusion peptide at the amino terminus of the resulting transmembrane subunit.

In this report, it is demonstrated that the spike glycoprotein of severe acute respiratory syndrome associated coronavirus requires processing by endosomal proteases at a site other than the predicted S1/S2 boundary on the way into the target cells. Furthermore, it appears that receptor-induced conformational rearrangements are necessary for this event to occur efficiently (see FIG. 7C). Cleavage at such a late stage of entry may preserve the structural integrity of S protein by preventing both premature triggering and/or dissociation of the receptor binding subunit (S1) from the membrane anchored S2 domain. A requirement for receptor-induced conformational rearrangements to expose the cleavage site would thus prevent precipitous activation during biosynthesis.

Typically, for viruses such as influenza, pH-dependence involves acid-induced conformational changes within the envelope glycoprotein allowing membrane fusion to occur. Expression of S protein on cells, however, allows cell-cell fusion at neutral pH, particularly when the receptor, ACE2, is overexpressed. Thus, S protein is able to mediate membrane fusion efficiently without any requirement for acid-mediated conformational change. Detectable cell-cell fusion using target cells expressing low endogenous levels of ACE2 required pretreatment with trypsin (Simmons et al., 2004, PNAS 101:4240-4245), leading us to hypothesize that the pH-dependence of infection may result from a requirement for cleavage by an acid protease within endosomes (Simmons et al., 2004, PNAS 101:4240-4245). In support of this hypothesis, infection of cells by mediated by SARS-CoV S protein was found to be blocked by a number of inhibitors of endosomal proteases, including specific inhibitors of cathepsin L. Moreover, cell-cell fusion with ACE2 overexpressing cells was reduced by pretreatment of the S protein effector cells with leupeptin (data not shown). Importantly, the requirement for proteolysis and involvement of cathepsin L were confirmed by the use of recombinant proteases in a cell-free model of S protein mediated membrane fusion. Both trypsin and the lysosomal protease, cathepsin L, were found to potently activate the fusion potential of S protein.

The requirement for a low pH component for S protein-mediated entry, highlighted by lysosomotropic agent sensitivity, can be explained by the pH-dependence of cathepsin L. A pH of less than or equal to 5.0 was found to be optimal for cathepsin L activation of S protein, while no activity was noted at neutral pH. Although the pH optimum for cathepsin L proteolysis in general is in the range pH 4.5-6.5, some studies have suggested that secreted cathepsin L is also active at neutral pH (Spiess et al., 1994, J Histochem Cytochem 42:917-929). Cathepsin L activity, however, is very much substrate specific. For example, the optimal pH for cathepsin L activation of urokinase-type plasminogen activator is pH 4.5, with over 10-fold less efficient activation occurring at pH 7.0 (Goretzki et al., 1992, L. FEBS Lett 297:112-118). Alternatively, the low pH requirement for cathepsin L activation may suggest that pH helps to expose the cleavage site in S. Arguing against this idea is the fact that trypsin can effectively replace cathepsins and activate S at neutral pH.

A number of lines of evidence suggest that interactions with ACE2 are required before efficient processing of S protein can occur. A model consistent with this data is that conformational rearrangements within S protein are induced by binding to receptor, thus exposing a normally inaccessible cleavage site (FIG. 7C). This is the first instance of a requirement for receptor-mediated conformational rearrangements prior to proteolytic cleavage of a viral fusion protein.

Overall, these experiments suggest a new paradigm for viral entry into target cells. Namely, that for SARS-CoV S protein, receptor-mediated conformational changes induce exposure of a cryptic cleavage site within viral envelope glycoprotein. Cleavage at this site by cellular proteases is then necessary in order to fully activate the viral glycoprotein's membrane fusion potential. Further characterization of this phenomena is likely to highlight steps in the activation of S protein that may yield targets for specific inhibitors of entry. Indeed, the finding that cathepsin L is an important activating protease for SARS infection indicates this as a target for therapeutic intervention. The entry process described here for SARS-CoV S protein also raises the question whether other classically defined pH-dependent viruses display this dependence due to a requirement for acidic protease activation and not pH-induced structural rearrangements as is commonly assumed. Future investigation will reveal if SARS-CoV represents the initial member of a new category of viral fusion proteins.

### Experimental Example 2

### Cathepsin L Inhibitor and Lentiviral Pseudotype System

Using the lentiviral pseudotype system described in Example 1, the effect of cathepsin L inhibitor MDL 28170 was assessed on viral entry of pseudotype virions having SARS CoV S glycoprotein. MDL 28170 was added at several different concentrations (0, 0.1, 0.3, 1, 3 and 10 micromolar) to mixtures of pseudotype virions and target cells.

The materials and methods used in the experiments presented in this Experimental Example are now described.

Cell Lines and Plasmids

Vero E6 cells (ATCC) were maintained in DMEM +5% fetal bovine serum (DMEM5). pCAGGS SARSCoV S, and plasmid for VSV-G have been previously described (Simmons et al, 2004, PNAS 101:4240).

Pseudotype Preparation

Pseudotypes were produced as previously described (Simmons et al, 2004, PNAS 101:4240). Briefly, 293T cells were transfected by calcium phosphate method with 10 pg of HIV gag/pol encoding luciferase (pNL-luc) (Connor et al, 1995, Virology 206:935) and 30 pg of pCAGGS SARS-CoV S or 15 fig of VSVG. Cells were washed the next day and then induced with sodium butyrate (10 mM) for 5 hours before further washing. Sixteen hours later, cell supernatant was filtered through a 0.45 μm pore size screen.

Protease Inhibitors

Vero E6 set-up overnight in 48-well plates at 2×104 cells/well. Cells were pre-treated for 1 hour with increasing concentrations of inhibitor in DMEM5. Inhibitors were removed and replaced with the same inhibitors at double the final concentration in 250 μl. An equal volume of HIV-luc(SARS S) or HIV-luc(VSV-G) were then added and cells were spin infected at 2000 rpm for 2 hours at 4° C. Following spin infection the cells were incubated for 5 hours, then medium was replaced with fresh DMEM5 without drug. Cells were assayed for luciferase activity after 40 hours as per manufacturer's instructions (Promega).

The results of the experiments presented in this Experimental Example are now described.

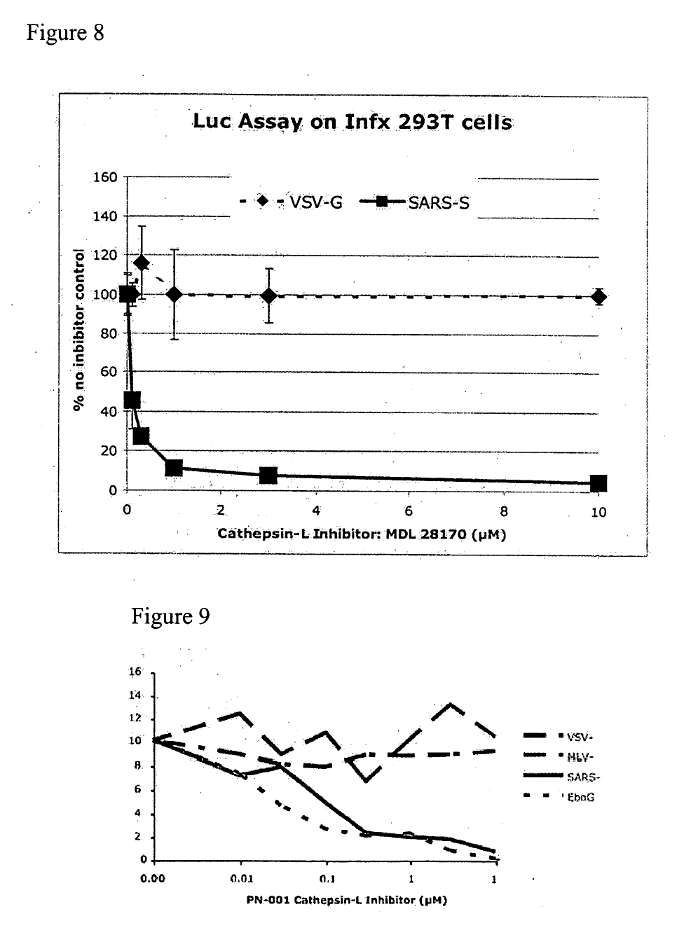

As shown in FIG. 8, MDL 28170 potently and specifically inhibits pseudotype infection mediated by SARS CoV S glycoprotein. Similarly, MDL 28170 potently and specifically inhibits pseudotype infection mediated by EboV GP glycoprotein, but not infection by viruses that do not require cathepsin L activity for viral entry (FIG. 9).

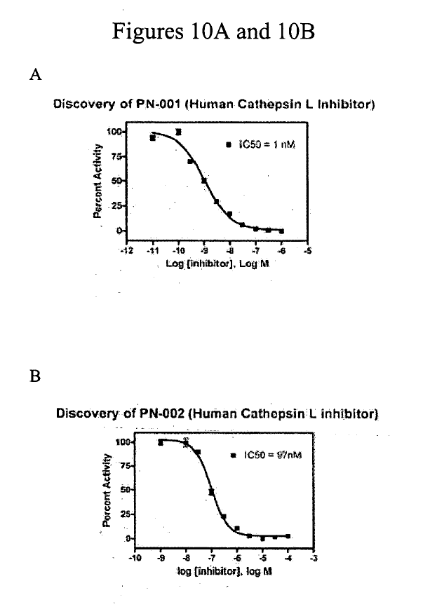

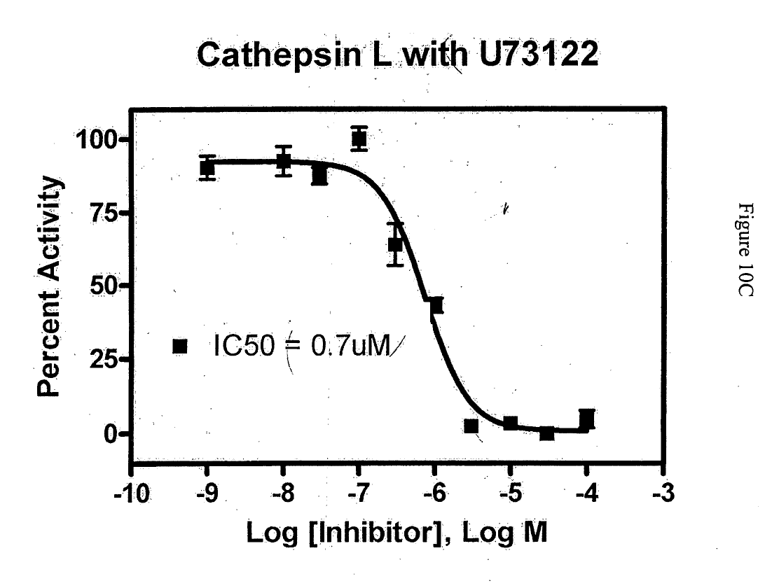

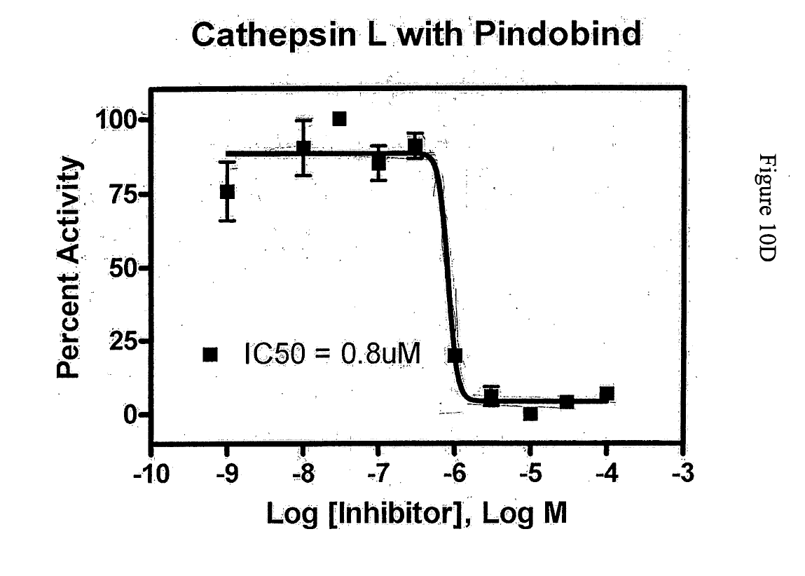

FIGS. 10A-10E show exemplary inhibition curves for a variety of compounds, (10A) PN-001; (10B) PN-002; (10C) U73122; (10D) pindobind; and (10E) 3,5 dinitro catechol. These data came from standard 384-well plate assays containing purified cathepsin L, a fluorogenic substrate (VEID-MCA) and the compound. As a result of using purified cathepsin L, the IC50 values obtained in this biochemical assay, compared to values determined by the lentiviral pseudotype cellular assay described above, are, as expected, smaller values. It is common that compounds are more potent in a pure biochemical assay compared to a cellular assay.

### Experimental Example 3

### Nanoliter Microarray Screening for Biochemical Assays

The materials and methods used in the experiments presented in this Experimental Example are now described.

Chemical compounds within individual nanoliter droplets of glycerol can be microarrayed onto glass slides at 400 spots/cm². Using aerosol deposition, subsequent reagents, and water are metered into each reaction center in order to rapidly assemble diverse multicomponent reactions without cross-contamination or the need for surface linkage. This proteomics technique allows for the kinetic profiling of protease mixtures, protease—substrate interactions, and high throughput screening reactions.

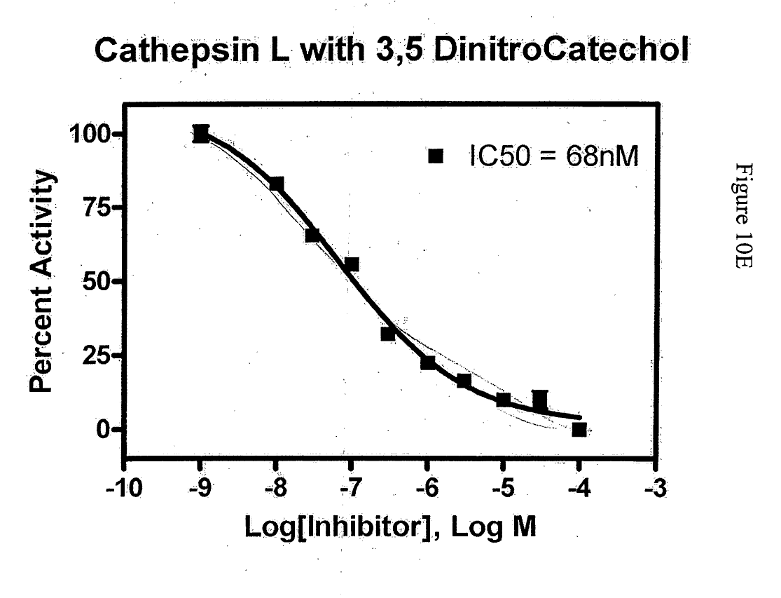

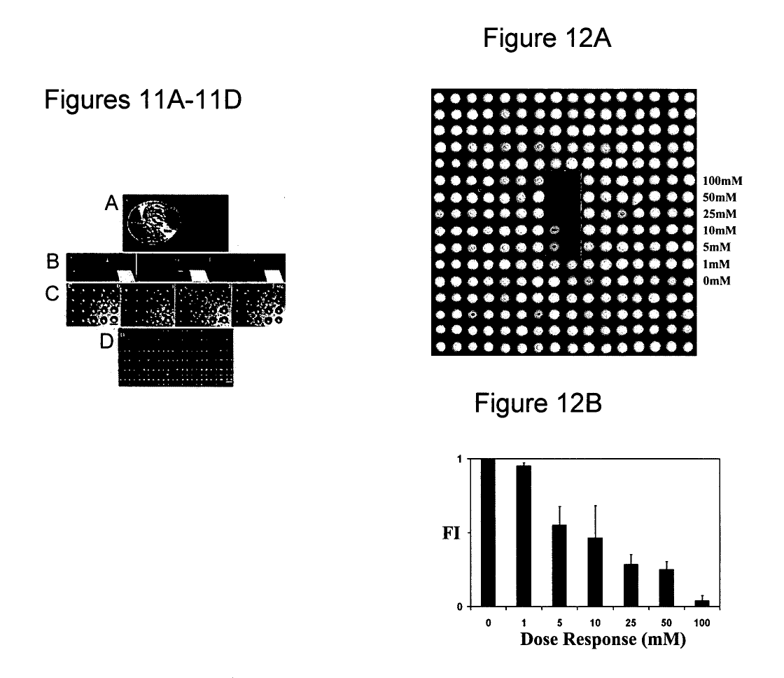

Exploiting the low volatility of glycerol droplets on glass, discrete reaction volumes are created via contact printing. Each droplet had an average volume of 1.6 nL after microarraying, as determined by imaging the spot height with calibrated differential interference contrast microscopy. A 16×24 array of 200 micrometer diameter spots with 500 micrometer center-to-center spacing, equivalent to a 384-well plate format, occupied less than 1-cm² (FIG. 11A). In kinetic applications on a microarray, the need to initiate tens of thousands of reactions at once is not easily accommodated by the use of piezo dispensing micropipettes or ink-jet engines that have exacting surface tension or viscosity requirements and are prone to clogging. To solve the problem of rapid sample delivery to these small nonspreading droplets, an aerosol generated with an ultrasonic nozzle operating at 120 kHz was deposited onto the arrays. In FIG. 11B, the aerosol was visualized under conditions of low carrier gas flow (<0.5 L/min). With carrier gas flow typical of actual use, 2.3 L/min, the aerosol was not visible by eye. In this approach, the aerosolization of the sample resulted in a fine mist with a median droplet diameter of 18 μm (˜3 pL). High speed imaging (400 frames per sec) of the aerosol revealed that the depositing aerosol was a turbulent and well-mixed helical vortex with a rotational frequency of ˜50-100 Hz. Aerosol droplets deposited evenly on and around the glycerol spots and rapidly evaporated within 7 seconds (FIG. 11C) without mixing between spots. The microarrays could be subjected to more than 10 separate depositions without spot mixing. Aerosol deposition allowed the pumping of water into the reaction center from 0.1 to 50% by volume required for enzymatic reactions to proceed.

To test the uniformity of the aerosol deposition and spot integrity, a microarray with alternating rows of 7-amino-4-methylcoumarin (AMC)-loaded spots and dye-free spots was subjected to aerosol deposition of tetramethylrhodamine-5-isothiocyanate (TRITC). Delivery of TRITC was uniform across the slide without cross-contamination of AMC rows (FIG. 11D) into the alternating rows of spots initially free of AMC. In an experiment with Tamara-Red delivered to ten 16×24 microarrays on 5 separate slides consisting of dye-free spots, the intraslide spot-to-spot coefficient of variation (CV) was ±16 % and the interslide average spot intensity CV was <3 %. Microarrays were stable at −20° C. for over 3 months.

### Experimental Example 4

### High Throughput Screening Using Nanoliter Microarray Screening

To demonstrate the feasibility of nanoliter scale screening assay of serine proteases, a 16×16 microarray of glycerol with the central positions (boxed in red, FIG. 12A) spiked with increasing doses from 0 to 100 mM of benzamidine, a thrombin inhibitor, was generated. Human thrombin at 10 U/ml (400 nL/s for 4 seconds) was deposited on the microarray, followed by deposition of boc-VPR-MCA at 10 mM in DMSO at 400 nL/s for 4 s. The microarray was incubated and imaged (FIG. 12A) to produce a dose-response curve with an IC50 of ˜5 mM (FIG. 12B) for benzamidine on thrombin in a high glycerol background. An IC50 of 5 mM was found at similar reaction conditions in glycerol in a 96-well plate assay. This value of the IC50 was somewhat higher than expected for benzamidine-mediated inhibition of thrombin in water (Kᵢ=0.3 to 1 mM). Hydrophobic association of glycerol with the thrombin surface may cause a reduction in the affinity of the inhibitor for thrombin. In experiments to detect other serine proteases, tissue plasminogen activator (tPA), urokinase, factor Xa, plasmin, and kallikrein were also easily detected with suitable MCA substrates in the microarray format.

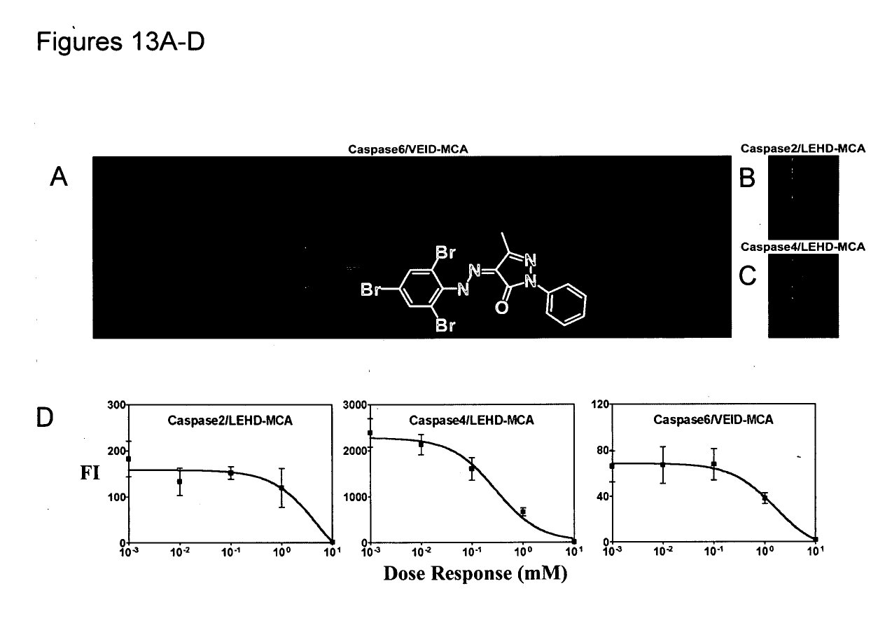

A commercially available exploratory library of 352 diverse compounds was microarrayed in 2 μL samples at 15 nmol/μL (l5mM in DMSO). In consuming about 1 nanomole of each compound, 100 replicate slides with each compound arrayed at 1 mM in glycerol in quadruplicate along with 32 blanks to serve as positive controls for uninhibited reaction were prepared. A microarray was then sprayed sequentially with human caspase 6 and then its substrate VEID-MCA. Four spots containing the same compound on the microarray displayed low substrate conversion (FIG. 13A). This compound, 5-methyl-2-phenyl-4-[(2,4,6-tribromo-phenyl)-hydrazono]-2,4-dihydro-pyrazol-3-one (compound 1, 515.0 MW), also reduced substrate conversion when microarrays were screened with human caspase 2 and LEHD-MCA (FIG. 13B) or human caspase 4 and LEHD-MCA (FIG. 13C). No inhibitors were detected with the 352-compound microarrays screened with human thrombin/boc-VPR-MCA or with bovine chymotrypsin/BODIPY-casein substrates. Compound 1 was then tested using a fluorescence plate reader for inhibitory activity in a standard background buffer (no glycerol). In these assays, compound 1 displayed an IC50 of about 5, 0.5, and 5 mM (FIG. 13D) against caspases 2, 4, and 6, respectively.

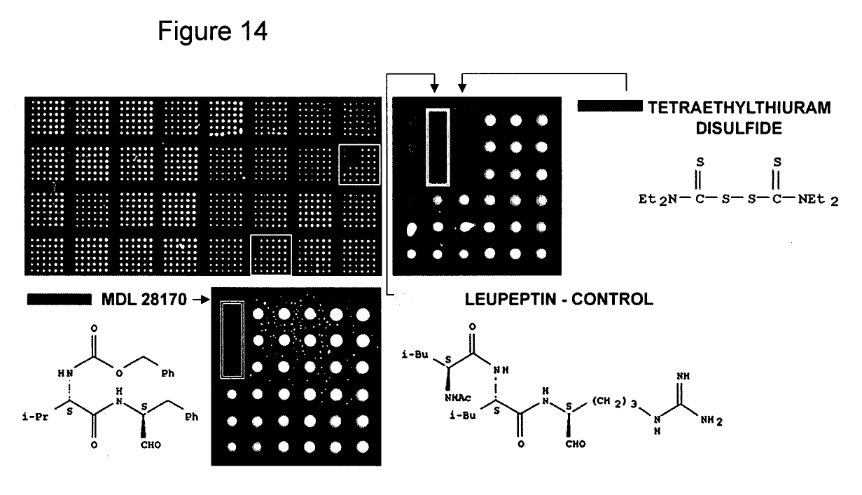

In a similar study, a test library (LOPAC) screened with cathepsin L revealed several hits in triplicate (FIG. 14), including MDL 28170, tetraethylthiuram disulfide and leupeptin (control inhibitor). Notably, MDL 28170 has not previously been identified as a cathepsin L inhibitor.

These data, therefore, demonstrate the usefulness of the nanoliter microarray HTS method for identification of cathepsin L inhibitors.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating or preventing a viral infection in an individual in need of such treatment, comprising administering an effective amount of at least one compound that is an inhibitor of cathepsin L.

2. The method of claim 1 wherein the viral infection comprises ebola or SARS.

3. The method of claim 2 wherein the compound is selected from the group consisting of Z-Val-Phe-CHO, Z-Val-Phe-FMK, Boc-Val-Phe-4-chlorobenzyl, Z-Val-Phe-NHO-enzyl, Z-Val-Phe-NHO-4-methoxybenzyl, and Z-Val-Phe-NHO-4-methylbenzyl.

4. The method of claim 2 wherein the compound is a compound according to Formula I: 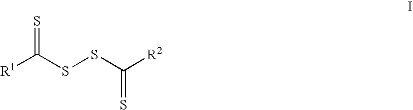
wherein R¹ and R² are independently selected from the group consisting of (C₃-C₁₀)hydrocarbyl and —N((C₁-C₆)alkyl)₂;
wherein —N((C₁-C₆)alkyl)₂ includes moieties wherein the two alkyl groups may combine to form a saturated heterocycle containing one nitrogen atom and from 4 to 7 carbon atoms.

5. The method of claim 4 wherein R¹ and R² are identical.

6. The method of claim 5, wherein R¹ and R² are selected from the group consisting of —N(Et)₂, cyclohexyl, cyclopentyl, cycloheptyl, phenyl, cyclopentadienyl, cyclobutyl and cyclopropyl.

7. The method of claim 6, wherein the thiuram disulfide compound is tetraethylthioperoxydicarbonic diamide,

8. The method of claim 2 wherein the compound is a compound according to Formula II: 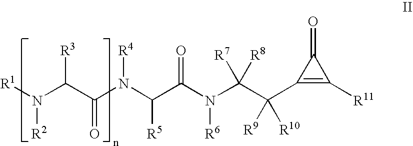
wherein:
wherein R¹ is —H, R¹²—CO—, R¹²—NH—CO— or R¹²—SO₂—;
wherein R¹² is C₁-C₂₀ alkyl optionally substituted by one or more substituents selected from the group consisting of C₃-C₁₅ cycloalkyl, optionally substituted C₆-C₁₄ aryl, C₃-C₁₅ cycloalkyloxy, optionally substituted C₆-C₁₄ aryloxy, optionally substituted C₆-C₁₄ arylthio, optionally substituted C₆-C₁₄ arylsulfonyl, optionally substituted C₇-C₂₀ aralkyloxy, optionally substituted heterocyclic, oxo, hydroxyl, C₁-C₁₀ alkoxycarbonyl group and carboxyl group; C₃-C₁₅ cycloalkyl; optionally substituted C₆-C₁₄ aryl and optionally substituted heterocyclic;
R², R⁴ and R⁶ each are independently —H or C₁-C₁₀ alkyl optionally substituted by C₁-C₅ alkoxy group or C₁-C₅ alkylthio group;
R₃, R₅ and R₇ each are independently —H, C₁-C₂₀ alkyl optionally substituted by C₃-C₁₀ cycloalkyl, C₃-C₁₀ cycloalkyl or optionally substituted C₇-C₂₀ aralkyl;
R⁸ is —H or C₁-C₂₀ alkyl;
R₇ and R⁶ taken together may form C₃-C₁₅ cycloalkyl group;
R⁹ is hydroxyl group or C₂-C₁₀ acyloxy;
R¹⁰ is —H;
R⁹ and R¹⁰ taken together may form oxo;
R¹¹ is —H, C₁-C₂₀ alkyl group optionally substituted by C₃-C₁₅ cycloalkyl, C₃-C₁₅ cycloalkyl, C₂-C₂₀ alkenyl group, optionally substituted C₆-C₁₄ aryl group, optionally substituted C₇-C aralkyl group, optionally substituted heterocyclic group or —C(R¹³)(R¹⁴)—OH, wherein R¹³ and R¹⁴ each are independently —H, C₁-C₂₀ alkyl, optionally substituted C₇-C₂₀ aralkyl or optionally substituted C₆-C₁₄ aryl, or R¹³ and R¹⁴ taken together may form C₃-C₁₅ cycloalkyl group); and n is 0 or 1, or pharmaceutically acceptable salts thereof.

9. The method of claim 2 wherein the compound is a compound according to Formula III: 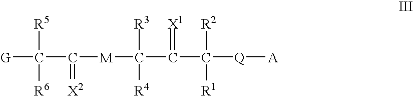
wherein:
M is O, NR⁷ or CR¹R²;
X¹ is O, S or NR⁷;
X² is O, S, NR⁷ or two H atoms, preferably O;
Q is O, S or NR¹;
R¹ and R² are independently —H, (C₁-C₁₀)alkyl, (C₁-C₁₀)heteroaryl, (C₁-C₁₀)alkanoyl, or aroyl, wherein the alkyl, heteroaryl, alkanoyl and aroyl groups are optionally substituted with J;
R³, R⁴, R⁵ and R⁶ are independently —H, (C₁-C₁₀)alkyl, aryl, or heteroaryl, wherein the alkyl, aryl and heteroaryl groups are optionally substituted with J;
R⁷ and R⁸ are independently —H, (C₁-C₁₀)alkyl, aryl, or heteroaryl, wherein the alkyl, aryl and heteroaryl groups are optionally substituted with J;
J is halogen, COOR⁷, R⁷OCO, R⁷OCONH, —OH, —CN, —NO₂, —NR⁷R⁸, N═C(R⁷)R⁸, N═C(NR⁷R⁸)₂, —SR⁷, —OR⁷, phenyl, naphthyl, heteroaryl, or —(C₃-C₈)cycloalkyl;
G is —NH₂, —NHR¹, —CH₂R¹, —CH₂C(O)B, carbobenzyloxy-NH, succinyl-NH, R⁷O-succinyl-NH, R⁷OC(O)NH, —CH₂ C(O)-(xanthen-9-yl), or —CH₂COR⁹;
R⁹ is an alkyl, aryl, or arylalkyl group of up to 13 carbons; or -AA¹-NHC(O)OCH₂C₆H₅;
wherein AA′ is one of the 20 natural amino acids or its opposite antipode;
B is (C₁-C₁₀)alkyl, (C₁-C₁₀)aralkyl, aryl having 1 to 3 carbocyclic rings, or heteroaryl having 1 to 3 rings, wherein the alkyl, aralkyl, aryl and heteroaryl groups are optionally substituted with J; and
A has the structure: 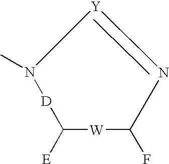
wherein: Y is N or CR¹;
W is a double bond or a single bond;
D is C═O or a single bond;
E and F are independently R¹, R², J, or when taken together E and F comprise an aliphatic carbocyclic ring having from 5 to 7 carbons, an aromatic carbocyclic ring having from 5 to 7 carbons, an aliphatic heterocyclic ring having from 5 to 7 atoms, or an aromatic heterocyclic ring having from 5 to 7 atoms;
wherein the aliphatic heterocyclic ring and the aromatic heterocyclic ring each have from 1 to 4,heteroatoms; and
the aliphatic carbocyclic ring, the aromatic carbocyclic ring, the aliphatic heterocyclic ring, and the aromatic heterocyclic ring are each optionally substituted with J.

10. The method of claim 2 wherein the compound is a compound according to Formula IV: 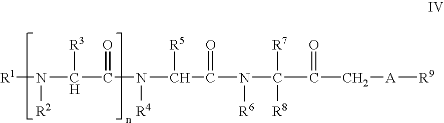
wherein:
R¹ is —H, R¹⁰—CO—, R¹⁰—O—CO—, R¹⁰—SO₂— or R¹⁰—NH—CO—;
wherein which R¹⁰ is C₁-C₂₀ alkyl optionally substituted by one or more substituents selected from the group consisting of C₃-C₁₅ cycloalkyl, C₃-C₁₅ cycloalkenyl, optionally substituted C₆-C₁₄ aryl, optionally substituted and partially hydrogenated C₁₀-C₁₄ aryl, fluorenyl, optionally substituted heterocyclic, C₃-C₁₅ cycloalkyloxy, optionally substituted C₆-C₁₄ aryloxy, optionally substituted and partially hydrogenated C₆-C₁₄ aryloxy, optionally substituted heterocyclic oxy, optionally substituted C₇-C₂₀ aralkyloxy and optionally substituted C₆-C₄ arylthio; C₃-C₁₅ cycloalkyl; optionally substituted C₆-C₁₄ aryl; optionally substituted and partially hydrogenated C₆-C₁₄ aryl; optionally substituted C₂-C₁₀ alkenyl and optionally substituted heterocyclic;
R², R⁴ and R⁶ each are independently —H or C₁-C₅ alkyl;
R³ and R⁵ each are independently —H, C₇-C₂₀ aralkyloxy, optionally substituted C₆-C₁₄ aryl, C₁-C₁₀ alkoxy or optionally substituted C₁-C₂₀ alkyl; wherein
R² and R³ and/or R⁴ and R⁵ taken together may form an optionally substituted nitrogen-containing heterocyclic ring;
R⁷ is C₁-C₂₀ alkyl optionally substituted by one or more substituents selected from the group consisting of C₃-C₁₅ cycloalkyl, hydroxyl, C₁-C₅ alkoxy optionally substituted by heterocyclic, C₆-C₁₄ aryloxy, C₇-C₂₀ aralkyloxy, C₁-C₅ alkylthio optionally substituted by heterocyclic, C₆-C₁₄ arylthio, C₇-C₂₀ aralkylthio, carboxyl, carbamoyl, C₂-C₆ alkoxycarbonyl, heterocyclic and optionally substituted C₆-C₁₄ aryl; —H; C₇-C₂₀ aralkyloxy; optionally substituted C₆-C₁₄ aryl and C₁-C₁₀ alkoxy;
R⁸ is —H, C₁-C₅ alkyl or optionally substituted C₇-C₂₀ aralkyl;
wherein R⁷ and R⁸ taken together may form optionally substituted benzylidene group or C₃-C₁5 cycloalkyl;
A is —S—, —SO—, —SO₂—, —O— or —N(R¹¹)—; wherein R¹¹ is —H or optionally substituted C₁-C₂₀ alkyl), and
(1) when A is —S—, —SO— or —SO₂—; then R⁹ is optionally substituted C₆-C₁₄ aryl group or —(CH₂)ₘ—X; wherein X is —H, —OH, C₁-C₅ alkylthio, C₂-C₆ alkoxycarbonylamino, optionally substituted heterocyclic, —NH₂, C₁-C₅ monoalkylamino, C₂-C₁₀ dialkylamino, C₂-C₆ acylamino, halogen, C₁-C₅ alkoxy, optionally substituted C₆-C₁₄ aryl or optionally substituted C₆-C₁₄ aryloxy; and
m is 0 or an integer of 1 to 15;
provided that if R¹ is benzyloxycarbonyl; then R⁴, R⁶ and R⁸ all are —H, R⁵ is benzyl, R⁷ is methyl and n is 0, then -A-R⁹ is not methylthio;
(2) when A is —O—, then R⁹ is —H or —(CH₂)₁—X; wherein 1 is an integer of 1 to 15; or
(3) when A is —N(R¹¹)—, then R⁹ is optionally substituted C₆-C₁₄ aryl or —(CH₂)ₘ—X; wherein X and m are as defined above; wherein R⁹ and R¹¹ taken together may form an optionally substituted nitrogen-containing heterocyclic ring; and
n is 0 or 1, or pharmaceutically acceptable salts thereof.

11. The method of claim 2 wherein the compound is a compound according to Formula V: 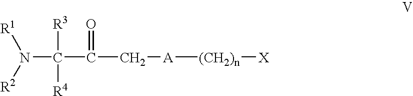
wherein:
R¹ is —H, 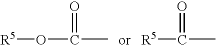
R⁵ is selected from the group consisting of C₁-C₂₀ alkyl, optionally substituted by one or more substituents selected from the group consisting of C₆-C₁₄ aryl optionally substituted by one or more substituents, fluorenyl, a heterocyclic residue optionally substituted by one or more substituents, C₃-C₁₅ cycloalkyl, C₃-C₁₅ cycloalkyloxy, C₆-C₁₄ aryloxy optionally substituted by one or more substituents, C₇-C₂₀ aralkyloxy optionally substituted by one or more substituents, C₆-CI₄ arylthio optionally substituted by one or more substituents, —OH, C₂-C₁₀ acyloxy; C₂-C₁₀ alkenyl optionally substituted by C₆-C₁₄ aryl optionally substituted by one or more substituents or by a heterocyclic residue optionally substituted by one or more substituents, C₆-C₁₄ aryl optionally substituted by one or more substituents; and a heterocyclic residue optionally substituted by one or more substituents;
R² and R⁴ are independently —H or C₁-C₅ alkyl,
R³ is —H, C₁-C₂₀ alkyl optionally substituted by one or more substituents, or C₆-C₁₄ aryl optionally substituted by one or more substituents, or when R³ and R⁴ are taken together, they are C₁-C₁₀ alkylene,
-A- is an oxygen atom, a sulfur atom or 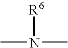
R⁶ is —H or C₁-C₅ alkyl;
n is an integer of from 1 to 10; and X is a heterocyclic residue optionally substituted by one or more substituents.

12. The method of claim 2 wherein the compound is a compound according to Formula VI: 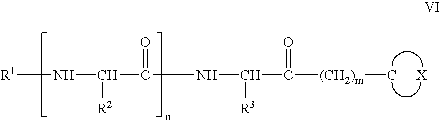
wherein: R¹ is —H,
R⁴ is C₁-C₂₀ alkyl optionally substituted by one or more substituents selected from the group consisting of C₃-C₁₅ cycloalkyl, C₆-C₁₄ aryl optionally substituted by one or more substituents, a heterocyclic residue optionally substituted by one or more substituents, C₃-C₁₅ cycloalkyloxy, C₆-C₁₄ aryloxy optionally substituted by one or more substituents, C₇-C₂₀ aralkyloxy optionally substituted by one or more substituents, and C₆-C₁₄ arylthio optionally substituted by one or more substituents; C₂-C₁₀ alkenyl optionally substituted by C₆-C₁₄ aryl optionally substituted by one or more substituents; C₆-C₁₄ aryl optionally substituted by one or more substituents; or a heterocyclic residue optionally substituted by one or more substituents,
R² and R³ are independently —H or C₁-C₂₀ alkyl optionally substituted by one or more substituents, 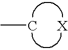
is a heterocyclic group containing at least one heteroatom, X, selected from the group consisting of N, S and O, which heterocyclic group is optionally substituted by one or more substituents selected from the group consisting of halogen and C₁-C₃ alkyl,
n is 0 or 1 and m is an integer of from 1 to 5.

13. The method of claim 2 wherein the compound is a compound according to Formula VIIa, VIIb, VIIc and VIId: 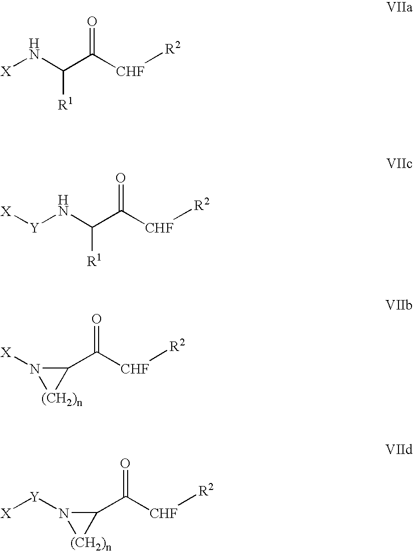
wherein:
R¹ and R² are independently selected from the —H; —C₁-C₆ alkyl; substituted alkyl of 1-6 carbons; aryl; aryl(C₁-C₄)alkyl;
n is an integer from 1-4 inclusive;
X is a peptide end-blocking group; and
Y is an amino acid or peptide chain of from 1-6 amino acids.

14. A method according to claim 2 wherein the compound is a compound according to Formula VIIIa or VIIIb: 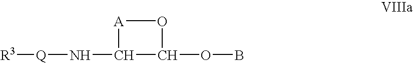
wherein:
Q is one or two amino acid residues which are optionally substituted;
R³ is a carboxyl group which are optionally esterified, or an acyl group;
A is an alkylene group;
B is —H, an alkyl group which may or may not be substituted or an acyl group, or a salt of the compound, and
Formulae VIIIb, is defined as: 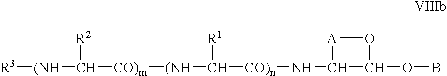
wherein:
R¹ and R² may be the same or different and each is —H or a hydrocarbon group which is optionally substituted;
R³ is a carboxyl group which is optionally esterified, or an acyl group;
A is an alkylene group;
B is —H, an optionally substituted alkyl group, or an acyl group;
m and n are independently 0 or 1, and may be the same or different; provided that where both m and n are equal to 0, R³ represents a carboxyl group which may be esterified or an acyl group having not less than 7 carbon atoms, or a salt of the compound.

15. The method of claim 2 wherein the compound is a compound according to Formula IX: 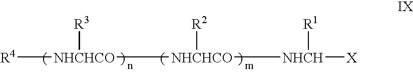
wherein:
R¹ is —H or an arylalkyl, heterocyclic-alkyl or an optionally substituted lower alkyl group;
R² and R³, independently are —H or an optionally substituted hydrocarbon residue;
R⁴ is an optionally substituted alkanoyl, sulfonyl, carbonyloxy, carbamoyl or thiocarbamoyl group;
X is —CHO or —CH₂OB (wherein B is —H or a protecting group of hydroxyl group); and
m and n independently are 0 or 1;
provided that R⁴ is an alkanoyl group substituted by aryl, a sulfonyl group substituted by aryl having more than 9 carbon atoms or by lower alkyl or a carbamoyl or thiocarbamoyl group which may be substituted when R¹ is an unsubstituted lower alkyl, arylalkyl or methylthiomethyl group; R² and R³ independently are a lower alkyl or arylalkyl group, X is —CHO, m is 1 and n is 0 or 1, or a salt thereof.

16. The method of claim 2 wherein the compound is a compound according to Formula X: 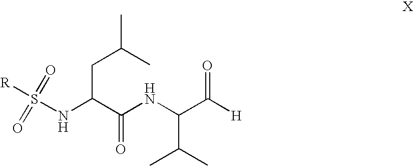
wherein:
R is C₆₋₁₄ aryl optionally substituted by one or more substituents selected from the group consisting of halogen; C₁₋₅ alkyl; trifluoromethyl; C₁₋₅ alkoxy; C₁₋₅ cyclic acetal residue; hydroxyl; C₂₋₆ acyloxy; formyl; carboxyl; C₂₋₆ alkoxycarbonyl; oxo; C₂₋₆ acyl; amino; C₁₋₅ monoalkylamino; C₂₋₁₀ dialkylamino; C₂₋₆ acylamino; carbamoyl; and C₂₋₆ alkylcarbamoyl or a heterocyclic residue having 1 to 4 hetero atoms selected from a group consisting of oxygen, sulfur and nitrogen and having, in total, 5 to 10 carbon atoms constituting a ring which is optionally substituted by one or more substituents selected from the group consisting of halogen; C₁₋₅ alkyl; trifluoromethyl; C₁₋₅ alkoxy; C₁₋₅ cyclic acetal residue; hydroxyl; C₂₋₆ acyloxy; formyl; carboxyl; C₂₋₆ alkoxycarbonyl; oxo; C₂₋₆ acyl; amino; C₁₋₅ monoalkylamino; C₂₋₁₀ dialkylamino; C₂₋₆ acylamino; carbamoyl; and C₂₋₆ alkylcarbamoyl.

17. The method of claim 2 wherein the compound is a compound according to Formula XI: 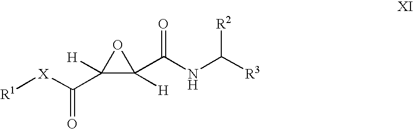
wherein:
R¹ is —H, a C₁-C₃₀ alkyl group, a C₆-C₄₀ aryl group, or a C₇-C₄₀ aralkyl group;
R² and R³ are independently selected from the group consisting of a C₆-C₄₀ aryl group, a C₇-C₂₀ aralkyl group, and a C₃-C₁₀ alkyl group;
X represents —O— or —NR⁴—; and R⁴ represents —H, a C₁-C₁₀ alkyl group, or a C₇-C₂₀ aralkyl group.

18. The method of claim 2 wherein the compound is a compound according to Formula XII: 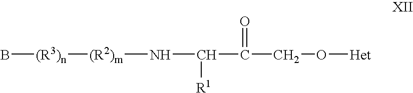
wherein:
B is —H or an amino acid blocking group for an N-terminal amino acid nitrogen;
R¹ is the amino acid side chain of the P₁ amino acid residue;
R² is the amino acid residue of the P₂ amino acid;
R³ is the amino acid residue of the P₃ amino acid;
n is 0 or 1;
m is 0 or 1; and
Het is the heterocyclic portion of the leaving group; wherein the heterocyclic leaving group includes a four-, five-, six- or seven-membered ring having at least one C and at least one of N, O or S in the ring.

19. The method of claim 2 wherein the compound is a polypeptide compound selected from the group consisting of SEQ ID NO. 1 and anti-cathepsin L antibodies.

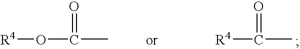

20. The method of claim 2 wherein the compound is a compound according to Formula XIII: 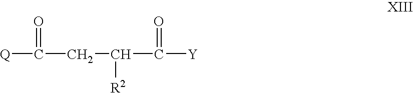
wherein:
Q is aryl having from 6 to 14 carbons, heteroaryl having from 6 to 14 ring atoms, aralkyl having from 7 to 15 carbons, alkyl having from 1 to 10 carbons, said alkyl groups being optionally substituted with one or more J groups, heteroalkyl having from 2 to 7 carbons, arylheteroalkyl wherein the aryl portion can be unfused or fused with the heteroalkyl ring, alkoxy having from 1 to 10 carbons, aralkyloxy having from 7 to 15 carbons, a carbohydrate moiety optionally containing one or more alkylated hydroxyl groups, xanthene-9-yl, CH(i-C₄H₉)NHCbz, CH₂N(i-C₄H₉)Cbz, or Formula XIIIa or XIIIb: 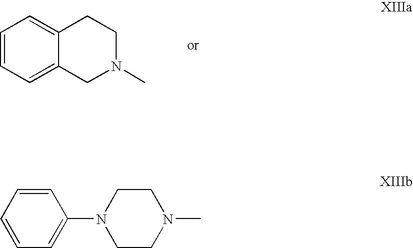
Y has the formula: 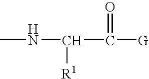
wherein: R¹ and R² are independently —H, alkyl having from one to about 14 carbons, cycloalkyl having from 3 to about 10 carbons, or a natural or unnatural side chain of an L-amino acid, said alkyl and cycloalkyl groups being optionally substituted with one or more J groups;
J is halogen, lower alkyl, aryl, heteroaryl, amino optionally substituted with one to three aryl or lower alkyl groups, guanidino, alkoxycarbonyl, aralkoxycarbonyl, alkoxy, hydroxy, or carboxy; and
G is —H, C(═O)NR³R⁴, C(═O)OR³ or CH₂R⁵;
wherein:
R³ and R⁴ are each independently —H, alkyl having from 1 to 10 carbons, said alkyl groups being optionally substituted with one or more J groups, aryl having from 6 to 14 carbons, and aralkyl having from 7 to 15 carbons; and
R⁵ is halogen;
with the proviso that if G is —H and Q is alkyl substituted with J, and said J is an α-amino group, then the α-amino nitrogen must be tertiary.

21. The method of claim 2 wherein the compound is a compound according to Formula XIV: 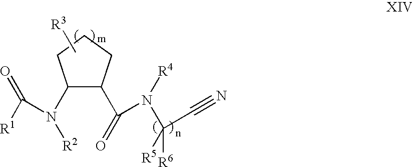
wherein:
m is 1, 2 or 3;
n is 1 or 2;
p is from 0 to 2;
R¹ is optionally substituted indolyl; optionally substituted indazolyl; optionally substituted benzothiazole; optionally substituted indolizinyl; optionally substituted tetrahydropyridoindolyl; optionally substituted pyridinylthiophenyl; or optionally substituted benzopyrrolothiazolyl;
R², R³, R⁴ and R⁵ each independently are —H or alkyl; and
R⁶ is —H; alkyl, cycloalkyl, or —(CR^{a}R^{b})_{q}-A;
wherein R^{a} and R^{b} each independently is —H or alkyl, q is from 0 to 3, and wherein A is
hydroxy, alkoxy, cyano, optionally substituted phenyl, optionally substituted pyridyl, optionally substituted imidazolyl, optionally substituted thienyl, —S(O)ᵣ—R^{c} wherein r is from 0 to 2 and R^{c} is —H or alkyl, —COR^{d}; wherein R^{d} is hydroxy, alkoxy, morpholinyl, or cycloalkylamino; or —NR^{e}R^{f} wherein R^{e} and R^{f} each independently is —H or alkyl, or R^{e} and R^{f} together with the nitrogen to which they are attached may form a five or six membered ring that optionally includes an additional heteroatom selected from O, N and S; and pharmaceutically acceptable salts, solvates or prodrugs thereof.

22. The method of claim 2 wherein the compound is selected from the group consisting of N—[N-(L-3-trans-carboxyoxirane-2-carbonyl)-L-leucyl]-agmatine, Leupeptin, Z-Phe-Gly-NHO-Bz, Z-Phe-Gly-NHO-Bz-pOMe, Z-Phe-Gly-NHO-Bz-pMe, Z-Phe-Phe-FMK, Z-Phe-Tyr-CHO, Z-Phe-Tyr(t-Bu)-diazomethylketone, 1-napthalenesulfonyl-Ile-Trp-CHO, Z-Phe-Tyr(OtBu)-COCHO, Z-LLL-FMK, Pindobind, L-3-carboxy-trans-2,3-epoxypropyl-leucylamide-(3-guanizino)butane, 1-(6-((8R,9S,13S,14S)-7,8,9,11,12,13,14,15,16,17-decahydro-3-methoxy-13-methyl-6H-cyclopenta[a]phenanthren-17-ylamino)hexyl)-1H-pyrrole-2,5-dione and 3,5 dinitrocatechol.

23. A method of identifying potential therapeutic compounds for treatment or prevention of SARS or Ebola viral infection, said method comprising the steps of:
assaying a test compounds using a lentiviral pseudotype system to assess viral entry in target cells,
wherein reduced viral entry in the presence of a test compound is indicative of a potential therapeutic compound for treatment or prevention of SARS or Ebola viral infection.

24. A method of identifying potential therapeutic compounds for treatment or prevention of SARS or Ebola viral infection, said method comprising the steps of:
assaying cathepsin L, a cathepsin L substrate and a test compound using a nanoliter microassay system,
wherein inhibition by a test compound of cathepsin L cleavage of the cathepsin L substrate is indicative of a potential therapeutic compound for the treatment or prevention of SARS or Ebola viral infection.

25. The method of claim 24, wherein said cathepsin L is human cathepsin L.

26. The method of claim 24, wherein said cathepsin L substrate is one of a fluorogenic substrate or a colorimetric substrate.
